(19) United States
(12) Patent Application Publication
(10) Pub. No.: US 2008/0124273 A1
(43) Pub. Date: May 29, 2008

(54) Novel cationic metal complex radiopharmaceuticals

(76) Inventors: Shuang Liu, West Lafayette, IN (US); Zhengjie He, Tianjin (CN)

Correspondence Address: BRINKS HOFER GILSON & LIONE, P.O. BOX 10395, CHICAGO, IL, 60610, US

(21) Appl. No.: 11/811,064

(22) Filed: Jun. 8, 2007

Related U.S. Application Data
(63) Continuation of application No. PCT/US2005/044597, filed on Dec. 8, 2005.
(60) Provisional application No. 60/634,357, filed on Dec. 8, 2004.

(51) Int. Cl. A61K 51/04; C07F 13/00

(52) U.S. Cl. 424/1.65; 534/14

(57) ABSTRACT

This invention relates to novel cationic crown-ether containing metal complexes, methods of preparing the cationic crown-ether containing metal complexes, and radiopharmaceutical compositions comprising the cationic crown-ether containing metal complexes. This invention relates particularly to cationic crown-ether containing ^{99m}Tc complex radiopharmaceuticals for diagnosis of cardiovascular disorders and cancer. This invention further relates to cationic crown-ether containing ^{186/188}Re complex radiopharmaceuticals for radiotherapy of cardiovascular disorders and cancer.

### FIELD OF THE INVENTION

This invention relates to novel crown-ether containing cationic metal complexes, methods of preparing the crown-ether containing cationic metal complexes, and radiopharmaceutical compositions comprising the crown-ether containing cationic metal complexes. This invention relates particularly to crown-ether containing cationic ^{99m}Tc complex radiopharmaceuticals for diagnosis of cardiovascular disorders and cancer. This invention further relates to crown-ether containing cationic ^{186/188}Re complex radiopharmaceuticals for radiotherapy of cardiovascular disorders and cancer.

### BACKGROUND OF THE INVENTION

Technetium-99m (^{99m}Tc) ligand complexes are well-known to be useful as imaging agents. The FDA has approved kits for the preparation of such complexes as ^{99m}Tc-Tetrofosmin [6,9-bis(2-ethoxyethyl)-3,12-dioxa-6,9-diphosphatetradecane ligands] as intravenous injection solutions used for the scintigraphic delineations of regions of reversible myocardial ischemia and ventricular function. Physical and metabolic properties of the coordinate ligands localized ^{99m}Tc-ligand imaging agents to specific organ tissues after intravenous injection. The resultant images can reflect organ structure or function. These images are obtained by means of a gamma camera that detects the distribution of ionizing radiation emitted by the radioactive molecules. Desirable agents and methods are those that minimize exposure to radioactive agents and maximize imaging resolution. Thus, superior heart-imaging agents adhere to myocardial tissue while at the same time have minimal affinity for other tissues and blood proteins. Ischemia-related diseases, particularly coronary artery disease (CAD), account for the majority of death in Western countries. Myocardial ischemia is a serious condition and the delay in reperfusion of the ischemic tissues can be life threatening. This is particularly true in the aged population. Rapid and accurate early detection of myocardial ischemia is highly desirable so that various therapeutic regiments can be given before irreversible myocardial damage occurs.

Myocardial perfusion imaging with radiotracers is an integral component of the clinical evaluation of patients with known or suspected coronary artery disease (CAD) in current clinical practice. The introduction of thallium-201 (²⁰¹Tl) in the mid 1970s was the turning point in the widespread clinical use of myocardial perfusion imaging, and had a profound impact on diagnostic evaluation, risk stratification, and therapeutic decision-making in patients with CAD over the last two decades. However, ²⁰¹Tl has its limitations. The vulnerability of ²⁰¹Tl to attenuation artifacts caused by the relatively lower energy emitted photons and lower count rate caused by the dose constraints may results in suboptimal images in a significant proportion of studies. In addition, ²⁰¹Tl images should be taken soon after injection, and may not be suitable for situations where immediate imaging may not be possible (for example, patients with acute myocardial infarction), mainly due to the dynamic nature of its distribution and redistribution dynamics. Therefore, there is a continuing effort in search of better radiopharmaceuticals for myocardial perfusion imaging.

Compared to ²⁰¹Tl, ^{99m}Tc yields relatively high-energy photons and can be used at much higher doses. The use of ^{99m}Tc also allows the simultaneous assessment of myocardial perfusion and cardiac function in a single study. Because of its ideal nuclear properties and its diverse coordination chemistry, ^{99m}Tc has been the isotope of choice for the development of myocardial perfusion imaging agents. Two cationic ^{99m}Tc complexes (^{99m}Tc-Sestamibi and ^{99m}Tc-Tetrofosmin) have been approved as commercial radiopharmaceuticals for myocardial perfusion imaging. Q3 and Q12 are cationic ^{99m}Tc complexes containing two monodentate phosphine ligands and a tetradentate Schiff-base chelator. Lipophilic ^{99m}Tc complexes, such as ^{99m}Tc-N-Noet, with neutral charge have also been studied for myocardial perfusion imaging. ^{99m}Tc-N-Noet is still under clinical investigation in Europe.

Perfusion is defined as blood flow at the cellular level—the delivery of nutrients and removal of waste products to maintain cellular function. An desirable myocardial perfusion agent should have a high first-pass extraction with stable myocardial retention, which linearly tracks myocardial blood flow over a wide range. Hepatic and gastrointestinal uptake should be minimal with exercise as well as with pharmacological stress and rest studies. The agent may redistribute; but should be in a predictable and reliable manner. Despite the widespread use of ^{99m}Tc-Sestamibi and ^{99m}Tc-Tetrofosmin in myocardial perfusion imaging studies, they do not meet the requirements of an ideal perfusion imaging agent mainly due to the low first-pass extraction and high uptake in liver and lungs. Therefore, there is still a continuing need for the development of better radiotracers for myocardial perfusion imaging. This invention is directed towards meeting this need. PNP6, EtOCH₂CH₂N[CH₂CH₂P(CH₂CH₂CH₂OEt)₂]₂ forms the complex ^{99m}TcN-DBODC6, [^{99m}Tc(N)(N(CH₂CH₂OEt)₂(PNP6)]⁺ with very low heart uptake and poor T/B ratios due to its high lipophilicity. Thus, the direct comparison of biodistribution characteristics of complexes with those of ^{99m}TcN-DBODC6, ^{99m}Tc-Sestamibi, and ^{99m}Tc-Tetrofosmin demonstrate superiority ligands for use in myocardial perfusion imaging agents.

### SUMMARY OF THE INVENTION

This invention relates to novel crown-ether containing cationic metal complexes, methods of preparing the crown-ether containing cationic metal complexes, and radiopharmaceutical compositions comprising the crown-ether containing cationic metal complexes. This invention relates particularly to crown-ether containing cationic ^{99m}TC complex radiopharmaceuticals for diagnosis of cardiovascular disorders and cancer, as well as other diseases. This invention further relates to crown-ether containing cationic ^{186/188}Re complex radiopharmaceuticals for radiotherapy of cardiovascular disorders, cancer, and other diseases. Accordingly,
[1] In a first embodiment the present invention provides a novel crown ether-containing cationic metal complex radiopharmaceutical of the formula:

L1-MC-L2
and pharmaceutically acceptable salt thereof, wherein
MC is the metal core, and is selected from a group of [M≡N]²⁺, [M=N═N—R¹]²⁺, [M=O]³⁺, and [M=N—R²]³⁺, wherein
M is the metallic radionuclide, and is selected from ^{99m}Tc, ^{94m}TC, ¹⁸⁶Re and ¹⁸⁸Re;
R¹ and R² can be the same or different, and are independently selected, at each occurrence, from the group consisting of: C₁₋₁₀ alkyl substituted with 1-5 R³, and aryl substituted with 1-4 R⁴ and 0-1 R⁵;
R³, R⁴ and R⁵ are independently selected, at each occurrence, from the group consisting of: H, F, Cl, Br, —OR⁶, —CO₂R⁶, —OC(═O)R⁶, —OC(═O)OR⁶, —OCH₂CO₂R⁶, —NR⁷C(═O)OR⁶, —SO₂R⁶, —SO₃R⁶, —NR⁷SO₂R⁶, and —PO₃R⁶;
R⁶ and R⁷ can be the same or different, and are independently selected, at each occurrence, from the group comprising of: C₁₋₁₀ alkyl, aryl group, and macrocyclic crown ether-containing group;
L1 is a bidentate ligand with a combination of O, N, P, and S donor atoms; and
L2 is a tridentate coligand with donor atoms such as phosphine-P, amine-N, and imine-N or a combination thereof
[2] A preferred embodiment of the present invention is a crown-ether containing cationic metal complex radiopharmaceutical of embodiment [1], wherein:
   MC is [M≡N]²⁺ or [M=N═N—R¹]²⁺;
   M is ^{99m}Tc or ^{94m}Tc;
   R¹ is selected from an aryl substituted with 1 or 2 R³;
   R³ is selected from the group consisting of: H, F, Cl, Br, —OR⁶, —CO₂R⁶, and —PO₃R⁶;
   R⁶ is selected from the group comprising of: C₁₋₅ alkyl and macrocyclic crown ether-containing group;
   L1 is a bidentate DTC chelator of the formula:

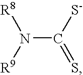

wherein R⁸ and R⁹ can be the same or different, and are independently selected, at each occurrence, from the group comprising of: H, C₁₋₁₀ alkyl, C₃₋₁₀ alkoxyalkyl, aryl, and macrocyclic crown ether-containing group, or R¹ and R² may be taken together to form a macrocycle of the formula [(CH₂)ₙ—O]_{b}—(CH₂)_{c}, wherein
a is 2-5;
b is 3-8;
c is 2-5;
L2 is tridentate bisphosphine coligand of the formula:

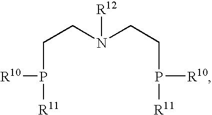

wherein R¹⁰ and R¹¹ can be the same or different, and are independently selected, at each occurrence, from the group comprising of: C₁₋₁₀ alkyl and alkoxyalkyl;
R¹² is selected from the group comprising of: C₁₋₁₀ alkyl substituted with 1-5 R¹³ and a macrocyclic crown ether-containing group; and
R¹³ is selected the group consisting of: —OR¹⁴, —CO₂R¹⁴, —CONR¹⁴R¹⁵, and —PO₃R¹⁴; and
R¹⁴ is R¹⁵ are C₁₋₁₀ alkyl.
[3] A more preferred embodiment of the present invention is a crown-ether containing cationic metal complex radiopharmaceutical of embodiment [2], wherein:
   R¹ is selected from an aryl substituted with a R³;
   R³ is selected from the group consisting of: H, Cl, —OR⁶, and —CO₂R⁶;
   R⁶ is selected from methyl or ethyl group;
   R⁸ and R⁹ can be the same or different, and are independently selected, at each occurrence, from the group comprising of: H, C₁₋₁₀ alkyl, C₃₋₅ alkoxyalkyl, and macrocyclic crown ether-containing group, or R¹ and R² may be taken together to form a macrocycle of the formula [(CH₂)ₐ—O]_{b}—(CH₂)_{c}, wherein
   a is 2 or 3;
   b is 3-6;
   c is 2 or 3;
   R¹⁰ and R¹¹ are can be the same or different, and are independently selected, at each occurrence, from the group comprising of: C₁₋₁₀ alkyl, C₃₋₁₀ alkoxyalkyl groups; and
   R¹² is an alkoxyalkyl group or a macrocyclic crown ether-containing group.
[4] A more preferred embodiment of the present invention is a crown ether-containing cationic metal complex radiopharmaceutical of embodiment [3], wherein:
   R⁸ and R⁹ are independently selected, at each occurrence, from the group comprising of: H, C₃₋₅ alkoxyalkyl, and macrocyclic crown ether-containing group, or R¹ and R² may be taken together to form a macrocycle of the formula [(CH₂)ₐ—O]_{b}—(CH₂)_{c}, wherein
   a is 2;
   c is 2;
   R¹⁰ and R¹¹ are can be the same or different, and are independently selected, at each occurrence, from the group comprising of C₁₋₁₀ alkyl, C₃₋₁₀ alkoxyalkyl groups; and
   R¹² is an alkoxyalkyl group or a macrocyclic crown ether-containing group.
[5] Another more preferred embodiment of the present invention is a crown ether-containing cationic metal complex radiopharmaceutical of embodiment [4], wherein L1 is selected from any one of the following crown-ether-containing chelator of the formula:

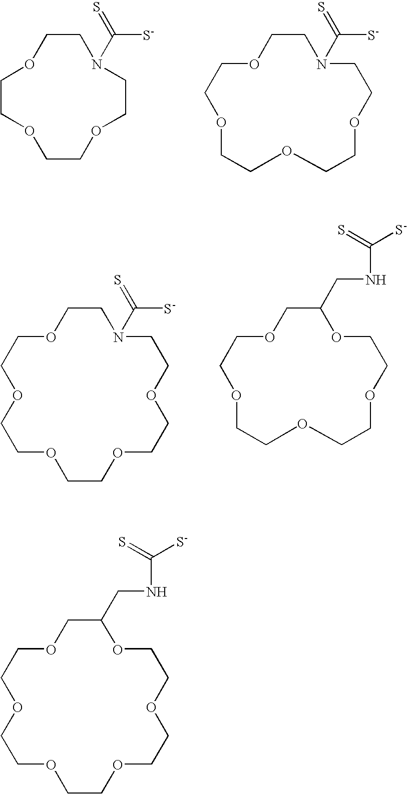

[6] Another more preferred embodiment of the present invention is a crown ether-containing cationic metal complex radiopharmaceutical of embodiment [4], wherein L2 is selected from any one of the following bisphosphine coligands of the formula:

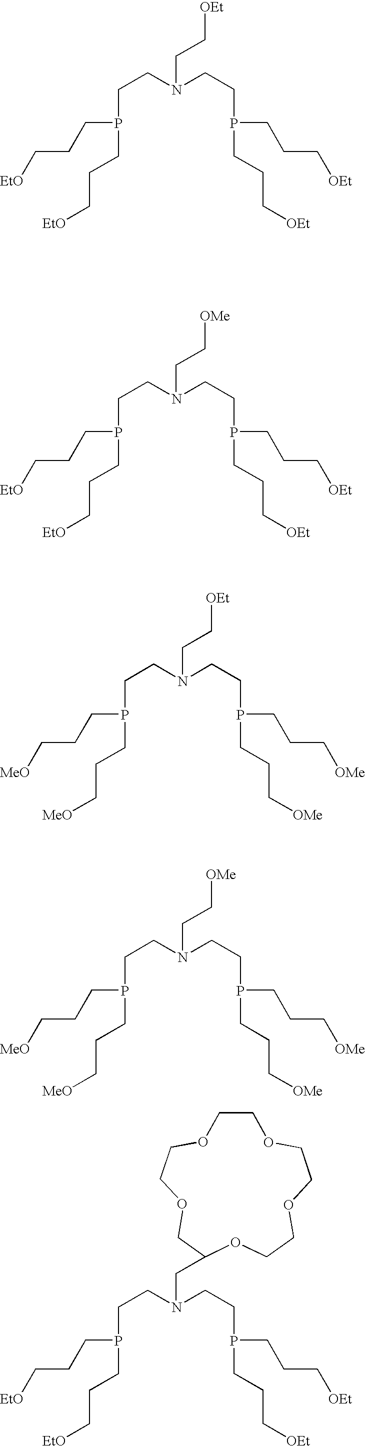

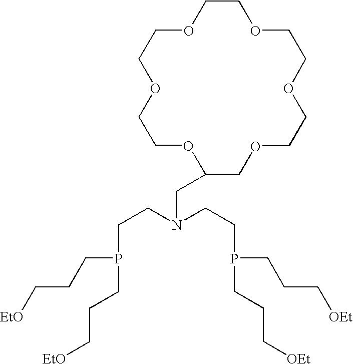

[7] Another more preferred embodiment of the present invention is a crown ether-containing cationic metal complex radiopharmaceutical of embodiment [4], wherein L1 is selected from any one of the following crown-ether-containing chelator of the formula:

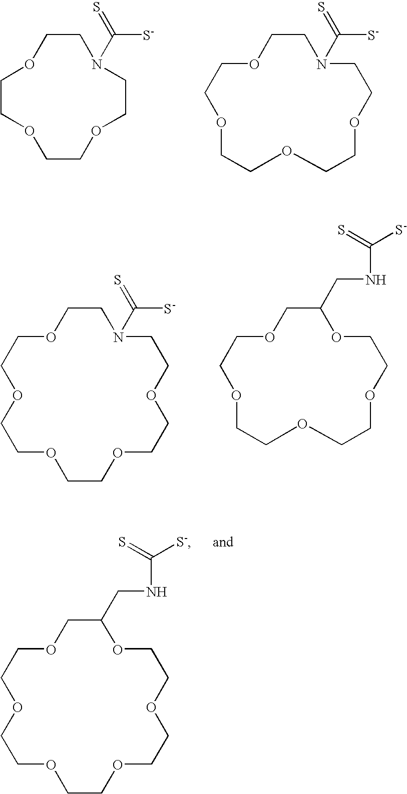

L2 is selected from any one of the following bisphosphine coligands of the formula:

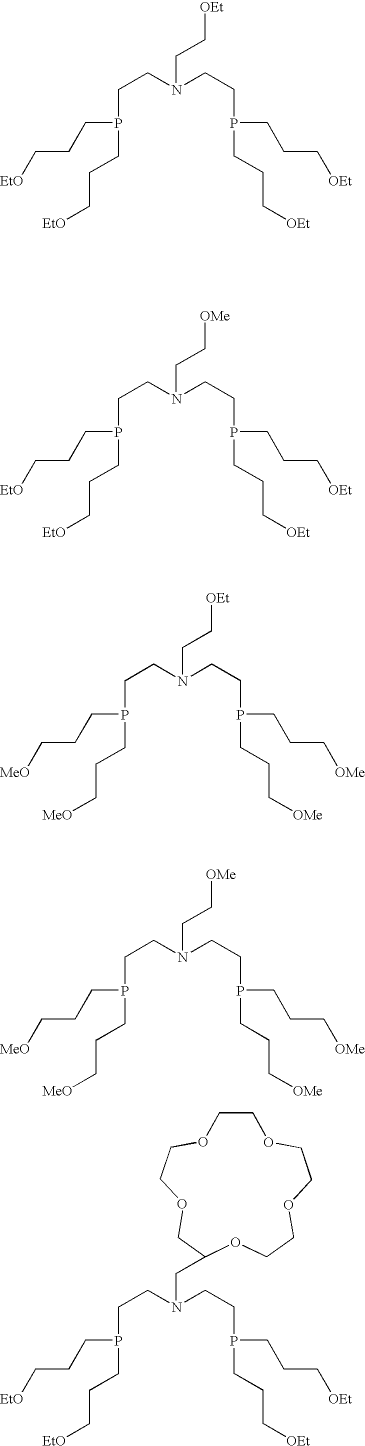

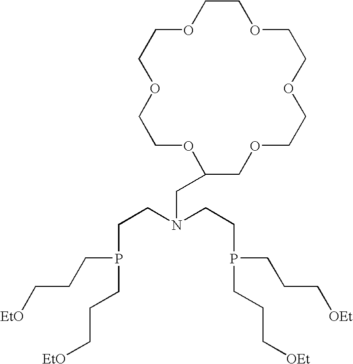

[8] Another preferred embodiment of the present invention is a novel radiopharmaceutical composition containing a crown ether-containing cationic metal complex radiopharmaceutical according to embodiments [1]-[7].
[9] Another preferred embodiment of the present invention is a method for preparation of a radiopharmaceutical product according to embodiments [1]-[7], comprising reacting pertechnetate with (1) a nitrido donor; (2) a reducing agent; (3) a crowned DTC chelator according to embodiments [1]-[7], and (4) a bisphosphine coligand according to embodiments [1]-[7].
[10] Another preferred embodiment of the present invention is a method according to embodiment [9], wherein the nitrido donor is succinyl dihydride, and the reducing agent is stannous chloride.
[11] Another preferred embodiment of the present invention is a kit for preparation of a radiopharmaceutical product according to embodiments [1]-[9], comprising:
   a first bottle containing a nitrido donor,
   a second bottle containing a stannous chloride and a chelating agent able to stabilize the tin cation,
   a third bottle containing a crowned DTC chelator according to embodiments [1]-[9]; and
   a fourth bottle containing a bisphosphine coligand according to embodiments [1]-[9].
[12] Another preferred embodiment of the present invention is a kit for preparation of a radiopharmaceutical product according to embodiment [11], comprising:
   a first bottle containing succinyl dihydride, a stannous chloride and a chelating agent able to stabilize the tin cation, and
   a second bottle containing a crowned DTC chelator according to embodiments [1]-[9]; and
   a third bottle containing a bisphosphine coligand according to embodiments [1]-[9].
[13] Another preferred embodiment of the present invention is a kit for preparation of a radiopharmaceutical product according to embodiment [12], comprising:
   a first bottle containing succinyl dihydride, stannous chloride and 1,2-diaminopropane-N,N,N′,N′-tetraacetic acid or a salt thereof, and
   a second bottle containing a crowned DTC chelator according to embodiments [1]-[9]; and
   a third bottle containing a bisphosphine coligand according to embodiments [1]-[9].
[14] Another preferred embodiment of the present invention is a method for preparation of a radiopharmaceutical product according to embodiments [1]-[7], comprising reacting pertechnetate with (1) a diazenido donor; (2) a reducing agent; (3) a crowned DTC chelator according to embodiments [1]-[7], and (4) a bisphosphine coligand according to embodiments [1]-[7].
[15] Another preferred embodiment of the present invention is a method for preparation of a radiopharmaceutical product according to embodiments [1]-[7], comprising reacting pertechnetate with (1) a diazenido donor; (2) a reducing agent; (3) a crowned DTC chelator according to embodiments [1]-[7], and (4) a bisphosphine coligand according to embodiments [1]-[7].
[16] Another preferred embodiment of the present invention is a method according to embodiment [15], wherein the diazenido donor is hydrazinobenzene, and the reducing agent is stannous chloride.
[17] Another preferred embodiment of the present invention is a kit for preparation of a radiopharmaceutical product according to embodiments [1]-[9], comprising:
   a first bottle containing hydrazinobenzene,
   a second bottle containing a stannous chloride and a chelating agent able to stabilize the tin cation,
   a third bottle containing a crowned DTC chelator according to embodiments [1]-[9]; and
   a fourth bottle containing a bisphosphine coligand according to embodiments [1]-[9].
[18] Another preferred embodiment of the present invention is a kit for preparation of a radiopharmaceutical product according to embodiment [17], comprising:
   a first bottle containing hydrazinobenzene, a stannous chloride and a chelating agent able to stabilize the tin cation, and
   a second bottle containing a crowned DTC chelator according to embodiments [1]-[9]; and
   a third bottle containing a bisphosphine coligand according to embodiments [1]-[9].
[19] Another preferred embodiment of the present invention is a kit for preparation of a radiopharmaceutical product according to embodiment [18], comprising:
   a first bottle containing hydrazinobenzene, stannous chloride and 1,2-diaminopropane-N,N,N′,N′-tetraacetic acid or a salt thereof, and
   a second bottle containing a crowned DTC chelator according to embodiments [1]-[9]; and
   a third bottle containing a bisphosphine coligand according to embodiments [1]-[9].
[20] Another preferred embodiment of the present invention is a novel radiopharmaceutical for radioimaging a mammal comprising (i) administering to said mammal an effective amount of a radiopharmaceutical of the formula according to embodiments [1]-[7], and (ii) scanning the mammal using a radioimaging device.
[21] In another preferred embodiment, the present invention provides a novel method for visualizing sites of myocardial disease in a mammal by radioimaging, comprising (i) administering to said mammal an effective amount of a radiopharmaceutical of formula according to embodiments [1]-[7], and (ii) scanning the mammal using a radioimaging device.
[22] In another preferred embodiment, the present invention provides a novel method of diagnosing a myocardial disease in a mammal comprising administering to said mammal a radiopharmaceutical composition of formula according to embodiments [1]-[7], and imaging said mammal.
[23] In another preferred embodiment, the present invention provides a novel method for visualizing sites of myocardial disease in a mammal by radioimaging, comprising (i) administering to said mammal an effective amount of a radiopharmaceutical of formula according to embodiments [1]-[7], and (ii) scanning the mammal using a radioimaging device.
[24] In another preferred embodiment, the present invention provides a novel method for visualizing sites of tumors in a mammal by radioimaging, comprising (i) administering to said mammal an effective amount of a radiopharmaceutical of formula according to embodiments [1]-[7], and (ii) scanning the mammal using a radioimaging device.
[25] In another preferred embodiment, the present invention provides a novel method for visualizing tumor multidrug resistance gene (MDR1) in a mammal by radioimaging, comprising (i) administering to said mammal an effective amount of a radiopharmaceutical of formula according to embodiments [1]-[7], and (ii) scanning the mammal using a radioimaging device.
[26] In another preferred embodiment, the present invention provides crown ether-containing cationic metal complex radiopharmaceutical of the formula:

L1-MC-L2
and pharmaceutically acceptable salt thereof, wherein
MC is the metal core, and is selected from a group of [M≡N]²⁺, [M=N═N—R¹]²⁺, [M=O]³⁺, and [M=N—R²]³⁺, wherein
M is the metallic radionuclide, and is selected from ^{99m}TC, ^{94m}TC, ¹⁸⁶Re and ¹⁸⁸Re;
R¹ and R² can be the same or different, and are independently selected, at each occurrence, from the group consisting of: C₁₋₁₀ alkyl substituted with 1-5 R³, and aryl substituted with 1-4 R⁴ and 0-1 R⁵;
R³, R⁴ and R⁵ are independently selected, at each occurrence, from the group consisting of: H, F, Cl, Br, —OR⁶, —CO₂R⁶, —OC(═O)R⁶, —OC(═O)OR⁶, —OCH₂CO₂R⁶, —NR⁷C(═O)OR⁶, —SO₂R⁶, —SO₃R⁶, —NR⁷SO₂R⁶ and —PO₃R⁶;
R⁶ and R⁷ can be the same or different, and are independently selected, at each occurrence, from the group comprising of: C₁₋₁₀ alkyl, aryl group, and macrocyclic crown ether-containing group;
L1 is a bidentate chelator of the formula:

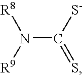

wherein R⁸ a substituted or unsubstituted macrocyclic crown ether-containing group attached to the nitrogen directly or through an alkyl or substituted alkyl group; and
R⁹ is H, C₁₋₁₀ alkyl, C₃₋₁₀ alkoxyalkyl, aryl, or macrocyclic crown ether-containing group
or R⁸ and R⁹ may be taken together to form a macrocycle of the formula [(CH₂)ₐ—O]_{b}—(CH₂)_{c}, wherein
a is 2-5;
b is 3-8;
c is 2-5;
; and
L2 is a tridentate bisphosphine coligand of the formula:

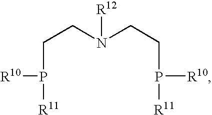

wherein R¹⁰ and R¹¹ can be the same or different, and are independently selected, at each occurrence, from the group comprising of: C₁₋₁₀ alkyl and alkoxyalkyl;
R¹² is selected from the group comprising of C₁₋₁₀ alkyl substituted with 1-5 R¹³ and a macrocyclic crown ether-containing group; and
R¹³ is selected the group consisting of: —OR¹⁴, —CO₂R¹⁴, —CONR¹⁴R¹⁵, and —PO₃R¹⁴; and
R¹⁴ is R¹⁵ are C₁₋₁₀ alkyl.
[27 ] In a first embodiment, the present invention provides a novel crown ether-containing cationic metal complex radiopharmaceutical of the formula

L1-MC-L2
and pharmaceutically acceptable salt thereof, wherein.
MC is the metal core, and is selected from a group of [M≡N]²⁺, [M=N═N—R¹]²⁺, [M=O]³⁺, and [M=N—R²]³⁺, wherein
M is the metallic radionuclide, and is selected from ^{99m}Tc, ^{94m}Tc, ¹⁸⁶Re and ¹⁸⁸Re;
R¹ and R² can be the same or different, and are independently selected, at each occurrence, from the group consisting of: C₁₋₁₀ alkyl substituted with 1-5 R³, and aryl substituted with 1-4 R⁴ and 0-1 R⁵;
R³, R⁴ and R⁵ are independently selected, at each occurrence, from the group consisting of H, F, Cl, Br, —OR⁶, —CO₂R⁶, —OC(═O)R⁶, —OC(═O)OR⁶, —OCH₂CO₂R⁶, —NR⁷C(═O)OR⁶, —SO₂R⁶, —SO₃R⁶, —NR⁷SO₂R⁶, and —PO₃R⁶;
R⁶ and R⁷ can be the same or different, and are independently selected, at each occurrence, from the group comprising of: C₁₋₁₀ alkyl, aryl group, and macrocyclic crown ether-containing group;
L1 is a bidentate chelator of the formula:

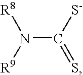

wherein R⁸ and R⁹ can be the same or different, and are independently selected, at each occurrence, from the group comprising of: H, C₁₋₁₀ alkyl, C₃₋₁₀ alkoxyalkyl, aryl, and macrocyclic crown ether-containing group, or
R¹ and R² may be taken together to form a macrocycle of the formula [(CH₂)ₐ—O]_{b}—(CH₂)c, wherein
a is 2-5;
b is 3-8;
c is 2-5
; and
L2 is a tridentate bisphosphine coligand of the formula:

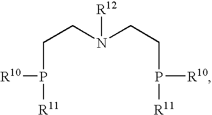

wherein R¹⁰ and R¹¹ can be the same or different, and are independently selected, at each occurrence, from the group comprising of C₁₋₁₀ alkyl and alkoxyalkyl;
R¹² is a substituted or unsubstituted macrocyclic crown ether-containing group attached to the nitrogen directly or through an alkyl or substituted alkyl group.
[28] In a preferred embodiment, the invention is a compound having the following formula:

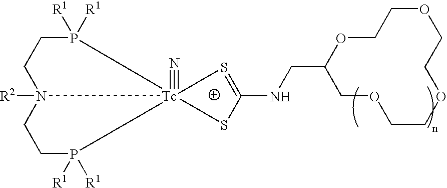

wherein R¹ is —(CH₂)₃OMe, —(CH₂)₃OEt, —(CH₂)₃OPropyl, —(CH₂)₃OButyl, —(CH₂)₃O(t)Butyl, or —(CH₂)₃OBenzyl;
R² is —(CH₂)OMe, —(CH₂)₂OEt, —(CH₂)₃OPropyl, —(CH₂)₃OButyl, —(CH₂)₃O(t)Butyl, —CH₂Ph, or —CH₂CH(OCH₂CH₂)ₘOCH₂CH₂;
n is 0 to 10; and

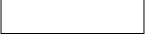

m is 1 to 10.
[29] In a preferred embodiment, the invention is a compound having the following formula:

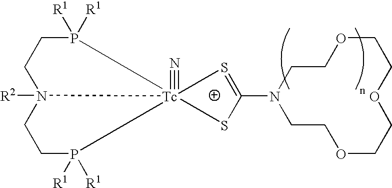

wherein R¹ is —(CH₂)₃OMe, —(CH₂)₃OEt, —(CH₂)₃OPropyl, —(CH₂)₃OButyl, —(CH₂)₃O(t)Butyl, or —(CH₂)₃O Benzyl;
R² is —(CH₂)₂OMe, —(CH₂)₂OEt, —(CH₂)₃OPropyl, —(CH₂)₃O Butyl, —(CH₂)₃O(t)Butyl, —CH₂Ph, or —CH₂CH(OCH₂CH₂)ₘOCH₂CH₂;
n is 0 to 10; and

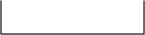

m is 1 to 10.
[30] In a preferred embodiment, the invention is a compound having the following formula:

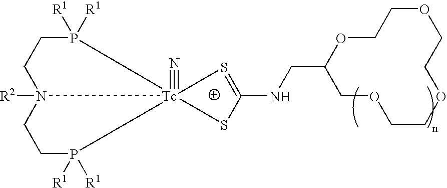

wherein R¹ is —(CH₂)₃OMe or —(CH₂)₃OEt;
R² is —(CH₂)₂OMe, —(CH₂)₂OEt, —CH₂Ph, or

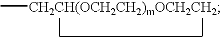

n is 2 or 3; and
m is 4 or 5.
[31] In a preferred embodiment, the invention is a compound having the following formula:

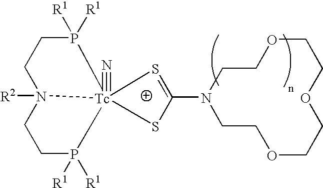

wherein R¹ is —(CH₂)₃OMe, —(CH₂)₃OEt;
R² is —(CH₂)₂OMe, —(CH₂)₂OEt, —CH₂Ph, or

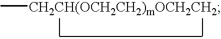

n is 1, 2, or 3; and
m is 4 or 5.

### DESCRIPTION OF THE FIGURES

FIG. 1. A typical HPLC chromatogram of [^{99m}TcN(3a)(L1d)]⁺.

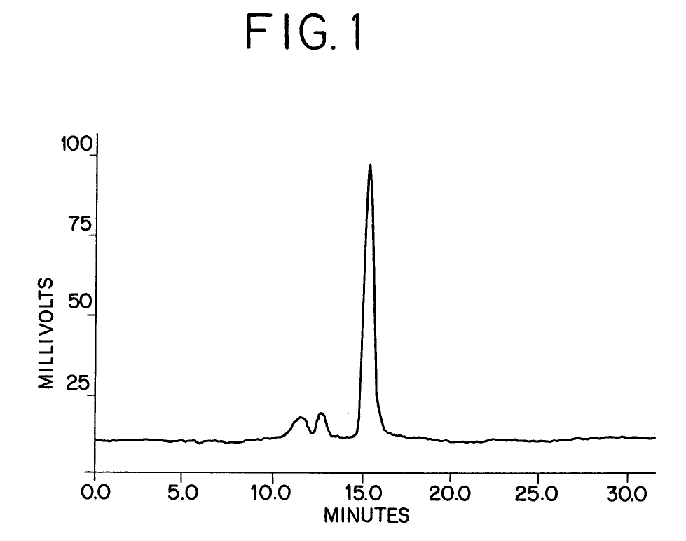

FIG. 2. Direct comparison of heart uptake between complexes [^{99m}Tc(3a)(DTC)]⁺ (DTC L1a−L1e) (DTC=L1-L5) and known heart imaging agents, ^{99m}Tc-Sestamibi, ^{99m}Tc-Tetrofosmin, ^{99m}TcN-DBODC5, and ^{99m}TcN-DBODC6, in the same animal model. Data for ^{99m}Tc-Sestamibi, ^{99m}Tc-Tetrofosmin, ^{99m}TcN-DBODC5 and ^{99m}TcN-DBODC6 were obtained from literature (Boschi, A. et al Nucl. Med. Commun. 2002, 23, and 689; Boschi, A. et al. J. Nucl. Med. 2003, 44: 806-814, both of which are herein incorporated by reference).

FIG. 3. Direct comparison of heart/liver ratios between complexes [^{99m}TcN(3a)(DTC)]⁺ (DTC=L1a−L1e) and known heart imaging agents, ^{99m}Tc-Sestamibi, ^{99m}Tc-Tetrofosmin, ^{99m}TcN-DBODC5, and ^{99m}TcN-DBODC6, in the same animal model. Data for ^{99m}Tc-Sestamibi, ^{99m}Tc-Tetrofosmin, ^{99m}TcN-DBODC5 and ^{99m}TcN-DBODC6 were obtained from literature (Boschi, A. et al Nucl. Med. Commun. 2002, 23, 689; Boschi A. et al. J. Nucl. Med. 2003, 44: 806-814).

FIG. 4. Direct comparison of heart/lung ratios between complexes [^{99m}TcN(3a)(DTC)]⁺ (DTC=L1a−L1e) and known heart imaging agents, ^{99m}Tc-Sestamibi, ^{99m}Tc-Tetrofosmin, ^{99m}TcN-DBODC5, and ^{99m}TcN-DBODC6, in the same animal model. Data for ^{99m}Tc-Sestamibi, ^{99m}Tc-Tetrofosmin, ^{99m}TcN-DBODC5 and ^{99m}TcN-DBODC6 were obtained from literature (Boschi, A. et al Nucl. Med. Commun. 2002, 23, 689; Boschi, A. et a). J. Nucl. Med. 2003, 44: 806-814).

### DEFINITIONS

The compounds herein described may have asymmetric centers. Compounds of the present invention containing an asymmetrically substituted atom may be isolated in, for example, optically active or racemic forms. It is well known in the art how to prepare optically active forms, such as by resolution of racemic forms or by synthesis from optically active starting materials. All chiral, diastereomeric, racemic forms and all geometric isomeric forms of a structure are intended, unless the specific stereochemistry or isomeric form is specifically indicated. All processes used to prepare compounds of the present invention and intermediates made therein are considered to be part of the present invention.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

When any variable (e.g., R⁶) occurs more than one time in any constituent or formula for a compound, its definition at each occurrence is independent of its definition at every other occurrence. Thus, for example, if a group is shown to be substituted with 0-2 R⁶, then said group may optionally be substituted with up to two R⁶ groups and R⁶ at each occurrence is selected independently from the definition of R⁶. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

As used herein, “alkyl” is intended to include both branched and straight-chain saturated aliphatic hydrocarbon groups having the specified number of carbon atoms. Examples of alkyl include, but are not limited to, methyl, ethyl, n-propyl, i-propyl, n-butyl, s-butyl, t-butyl, n-pentyl, and s-pentyl. “Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl. “Alkoxy” represents an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. “Cycloalkyl” is intended to include saturated ring groups, such as cyclopropyl, cyclobutyl, or cyclopentyl. Alkenyl” is intended to include hydrocarbon chains of either a straight or branched configuration and one or more unsaturated carbon-carbon bonds which may occur in any stable point along the chain, such as ethenyl and propenyl. “Alkynyl” is intended to include hydrocarbon chains of either a straight or branched configuration and one or more triple carbon-carbon bonds which may occur in any stable point along the chain, such as ethynyl and propynyl.

As used herein, the term “heterocycle” or “heterocyclic system” is intended to mean a stable 5- to 7-membered monocyclic or bicyclic or 7- to 10-membered bicyclic heterocyclic ring which is saturated partially unsaturated or unsaturated (aromatic), and which consists of carbon atoms and from 1 to 4 heteroatoms independently selected from the group consisting of N, O and S and including any bicyclic group in which any of the above-defined heterocyclic rings is fused to a benzene ring. The nitrogen and sulfur heteroatoms may optionally be oxidized. The heterocyclic ring may be attached to its pendant group at any heteroatom or carbon atom which results in a stable structure. The heterocyclic rings described herein may be substituted on carbon or on a nitrogen atom if the resulting compound is stable. A nitrogen in the heterocycle may optionally be quaternized. It is preferred that when the total number of S and O atoms in the heterocycle exceeds 1, then these heteroatoms are not adjacent to one another. It is preferred that the total number of S and O atoms in the heterocycle is not more than 1. As used herein, the term “aromatic heterocyclic system” or “heteroaryl” is intended to mean a stable 5- to 7-membered monocyclic or bicyclic or 7- to 10-membered bicyclic heterocyclic aromatic ring which consists of carbon atoms and from 1 to 4 heterotams independently selected from the group consisting of N, O and S. It is preferred that the total number of S and O atoms in the aromatic heterocycle is not more than 1.

Examples of heterocycles include, but are not limited to, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperazinyl, piperidinyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2-triazolyl, 1,3,4-triazolyl, and xanthenyl. Preferred heterocycles include, but are not limited to, pyridinyl, furanyl, thienyl, pyrrolyl, pyrazolyl, pyrrolidinyl, imidazolyl, indolyl, benzimidazolyl, 1H-indazolyl, oxazolidinyl, benzotriazolyl, benzisoxazolyl, oxindolyl, benzoxazolinyl, and isatinoyl. Also included are fused ring and spiro compounds containing, for example, the above heterocycles.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the present invention can, for example, be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

Since prodrugs are known to enhance numerous desirable qualities of pharmaceuticals (e.g., solubility, bioavailability, manufacturing, etc. . . . ) the compounds of the present invention may be delivered in prodrug form. Thus, the present invention is intended to cover prodrugs of the presently described compounds, methods of delivering the same and compositions containing the same. “Prodrugs” are intended to include any covalently bonded carriers which release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent. The coordination sphere of the radionuclide includes all the ligands or groups bound to the radionuclide. For a transition metal radionuclide to be stable it typically has a coordination number (number of donor atoms) comprised of an integer greater than or equal to 4 and less than or equal to 7; that is there are 4 to 7 atoms bound to the metal and it is said to have a complete coordination sphere. The requisite coordination number for a stable radionuclide complex is determined by the identity of the radionuclide, its oxidation state, and the type of donor atoms.

### DETAILED DESCRIPTION OF THE INVENTION

This invention relates to novel crown-ether containing cationic metal complexes, methods of preparing the crown-ether containing cationic metal complexes, and radiopharmaceutical compositions comprising the crown ether containing cationic metal complexes. This invention relates particularly to crown-ether containing cationic ^{99m}Tc complex radiopharmaceuticals for diagnosis of cardiovascular disorders and cancer. This invention further relates to crown-ether containing cationic ^{186/188}Re complex radiopharmaceuticals for radiotherapy of cardiovascular disorders and cancer.

The applicants have identified a number of preferred ^{99m}Tc-ligand complexes that contain cyclic ethylene glycol functional groups known as crown ethers. Several preferred ^{99m}Tc dithiocarbamate bidentate ligands of the current disclosure can be described as having the formula:

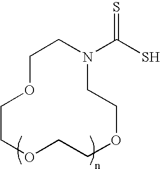

wherein n is greater than 1. Several other preferred ^{99m}Tc dithiocarbamate bidentate ligands of the current disclosure can be described as having the formula:

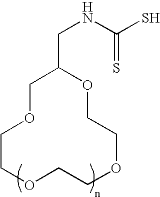

wherein n is greater than 1.

In one preferred embodiments of the current invention the ^{99m}Tc-ligand complex is a compound having the following formula:

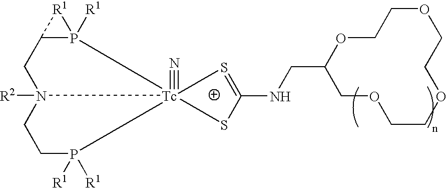

wherein R¹ is —(CH₂)₃OMe; R² is —(CH₂)₂OMe, —(CH₂)OEt, —CH₂Ph, or

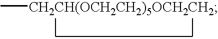

and n is 2 or 3.

The metallic radionuclide, M, may be selected from the group: ^{99m}Tc, ^{94m}Tc, ¹⁸⁶Re and ¹⁸⁸Re (or may be another metallic radionuclide). For diagnostic purposes ^{99m}Tc is generally the preferred isotope. Its 6 hour half-life and 140 keV gamma ray emission energy are almost ideal for gamma scintigraphy using equipment and procedures well established for those skilled in the art. The rhenium isotopes also have gamma ray emission energies that are compatible with gamma scintigraphy, however, they also emit high energy beta particles that are more damaging to living tissues. These beta particle emissions can be utilized for therapeutic purposes, for example, cancer radiotherapy. The related chemistry, medical applications, and radiolabeling with ^{186/188}Re by direct and indirect methods have been reviewed and the following articles are hereby incorporated by reference: Fritzberg, et al. Pharmaceutical Res. 1988, 5, 325; Liu et al. Bioconjugate Chem. 1997, 8, 621; Dilworth, J. R. and Parrott, S. J. Chem. Soc. Rev. 1998, 27, 43.

A radiopharmaceutical composition usually contains the metal complex radiopharmaceutical, a buffer, a stabilization aid to prevent autoradiolysis, and a bacteriostat. If radiopharmaceutical is prepared using the kit formulation, the radiopharmaceutical composition may contain the metal complex radiopharmaceutical and kit components, including unlabeled chelator, excess stabilizing coligand, a reducing agent, buffer, lyophilization aid, stabilization aid, solubilizing aids, and bacteriostats.

Buffers useful in the preparation of radiopharmaceuticals and in diagnostic kits useful for the preparation of said radiopharmaceuticals include but are not limited to phosphate, citrate, sulfosalicylate, and acetate. A more complete list can be found in the United States Pharmacopeia.

Lyophilization aids useful in the preparation of diagnostic kits useful for the preparation of radiopharmaceuticals include but are not limited to mannitol, lactose, sorbitol, dextran, Ficoll, and polyvinylpyrrolidine (PVP).

Stabilization aids useful in the preparation of radiopharmaceuticals and in diagnostic kits useful for the preparation of said radiopharmaceuticals include but are not limited to ascorbic acid, cysteine, monothioglycerol, sodium bisulfite, sodium metabisulfite, gentisic acid, ascorbic acid, and inositol.

Solubilization aids useful in the preparation of radiopharmaceuticals and in diagnostic kits useful for the preparation of said radiopharmaceuticals include but are not limited to ethanol, glycerin, polyethylene glycol, propylene glycol, polyoxyethylene sorbitan monooleate, sorbitan monoloeate, polysorbates, poly(oxyethylene)poly(oxypropylene)poly(oxyethylene) block copolymers (Pluronics) and lecithin. Preferred solubilizing aids are polyethylene glycol, and Pluronics.

Bacteriostats useful in the preparation of radiopharmaceuticals and in diagnostic kits useful for the preparation of said radiopharmaceuticals include but are not limited to benzyl alcohol, benzalkonium chloride, chlorbutanol, and methyl, propyl or butyl paraben.

A component in a diagnostic kit can also serve more than one function. A reducing agent can also serve as a stabilization aid, a buffer can also serve as a transfer ligand, a lyophilization aid can also serve as a transfer, ancillary or coligand and so forth.

The predetermined amounts of each component in the formulation are determined by a variety of considerations that are in some cases specific for that component and in other cases dependent on the amount of another component or the presence and amount of an optional component. In general, the minimal amount of each component is used that will give the desired effect of the formulation. The desired effect of the formulation is that the practicing end user can synthesize the radiopharmaceutical and have a high degree of certainty that the radiopharmaceutical can be safely injected into a patient and will provide diagnostic information about the disease state of that patient.

The diagnostic kits of the present invention may also contain written instructions for the practicing end user to follow to synthesize the radiopharmaceuticals. These instructions may be affixed to one or more of the vials or to the container in which the vial or vials are packaged for shipping or may be a separate insert, termed the package insert.

Radiopharmaceuticals are drugs containing a radionuclide, and are used routinely in nuclear medicine department for the diagnosis or therapy of various disease. They are mostly small organic or inorganic compounds with definite composition. They can also be macromolecules such as antibodies and antibody fragments that are not stoichiometrically labeled with a radionuclide. Radiopharmaceuticals form the chemical basis for nuclear medicine, a group of techniques used for diagnosis and therapy of various diseases. The in vivo diagnostic information is obtained by intravenous injection of the radiopharmaceutical and determining its biodistribution using a gamma camera. The biodistribution of the radiopharmaceutical depends on the physical and chemical properties of the radiopharmaceutical and can be used to obtain information about the presence, progression, and the state of disease.

In general, a radiopharmaceutical can be divided into two parts: the radiometal core and one or more organic chelators that coordinate strongly to the radiometal. The radiometal is the radiation source, and the selection of radiometal depends on the intended medical use (diagnostic or therapeutic) of the radiopharmaceutical. The organic chelator is used to stabilize the metallic core. The use of metallic radionuclides offers many opportunities for designing new radiopharmaceuticals by modifying the coordination environment around the metal with a variety of chelators. The coordination chemistry of the metallic radionuclide will determine the geometry and solution stability of the metal complex. Different metallic radionuclides have different coordination chemistries, and require chelators with different donor atoms and chelator frameworks. The biodistribution characteristics of the radiopharmaceutical are exclusively determined by chemical and physical properties of the radiometal complex. Therefore, the design of organic chelators is very important for the development of new radiopharmaceuticals for imaging and radiotherapy of various diseases, such as cardiovascular disorders, infectious disease and cancer.

One aspect of this invention relates to novel cationic crown ether-containing ^{99m}Tc complexes comprising a ^{99m}Tc core with two different ligands bonding to the Tc center. The ^{99m}Tc core can be [(^{99m}Tc≡N]²⁺, [^{99m}Tc═O]³⁺, [^{99m}Tc═N═N-aryl]²⁺, or [^{99m}Tc═N═N-aryl]²⁺. All these technetium cores have been used for preparation of ^{99m}Tc complex radiopharmaceuticals and the following articles are hereby incorporated by reference: Hom, R. K. and Katzenellenbogen, J. A. Nucl. Med. Biol. 1997, 24, 485; Dewanjee, M. K. Semin. Nucl. Med. 1990, 20, 5; Jurisson, et al Chem. Rev. 1993, 93, 1137; Dilworth, J. R. and Parrott, S. J. Chem. Soc. Rev. 1998, 27, 43; Liu, et al Bioconj. Chem. 1997, 8, 621; Griffiths, et al Bioconj. Chem. 1992, 3, 91; Liu, et al Chem. Rev. 1999, 99, 2235. The bidentate ligand may be neutral, monoanionic or dianionic with a combination of O, N, P, and S donor atoms, and is preferably crown-ether-containing dithiocarbamates (crowned DTCs). The tridentate coligand may be neutral, monoanionic or dianionic with donor atoms such as phosphine-P, anime-N, and imine-N or a combination thereof, and is preferably tridentate bisphosphines with a crown-ether moiety. It is preferred that at least one of the two ligands contains a crown ether group for improvement of heart or tumor uptake, and radioactivity clearance from blood, liver, and lungs.

The radionuclide for a diagnostic radiopharmaceutical is often a gamma-emitting isotope for scintigraphic imaging or positron-emitting isotope for positron emission tomography (PET). The choice of the radionuclide depends largely on the physical and nuclear properties (half-life and γ-energy), availability, and cost. Nearly 80% of all radiopharmaceuticals used in nuclear medicine department are ^{99m}Tc-labeled compounds. The 6 h half-life is long enough to allow a radiochemist to carry out radiopharmaceutical synthesis and for nuclear medicine practitioners to collect useful images. At the same time, it is short enough to permit the administration of millicurie amounts of ^{99m}Tc radioactivity without significant radiation dose to the patient. The monochromatic 140 KeV photons are readily collimated to give images of superior spatial resolution. Furthermore, ^{99m}Tc is readily available from commercial ⁹⁹Mo-^{99m}Tc generators at low cost.

^{99m}Tc may be produced from a parent radionuclide, ⁹⁹Mo, a fission product with a half-life of 2.78 days. In a ⁹⁹Mo—^{99m}Tc generator, [⁹⁹Mo]molybdate is absorbed to an alumina column and ^{99m}Tc is formed by decay of ⁹⁹Mo. The ^{99m}Tc in the form of [^{99m}Tc] pertechnetate is eluted from the column with saline. The ^{99m}Tc produced by the generator is not carrier-free because fifteen percent of ⁹⁹Mo decays directly to the long-lived isotope ⁹⁹Tc (t_{1/2}2.13×10⁵ y), which is also the single decay product of ^{99m}Tc. The specific activity of eluted ^{99m}Tc is very high and is dependent upon the prior-elution time. In general, the total concentration of technetium (^{99m}Tc and ^{99m}Tc) in the ⁹⁹Mo—^{99m}Tc generator eluent is in the range of 10⁻⁷-10⁻⁶ M.

For the last two decades, PET imaging was only used for academic research, most likely due the short half-life of isotopes, availability of generator systems, practicality of isotope production, transportation and distribution of the radiotracer. The development of outside vendors who can supply PET isotopes to a number of local customers on a unit dose basis and the adaptability of SPECT cameras for PET imaging should increase the use of this imaging modality. Compared to other imaging modalities, PET has the following three important technological features which enables clinicians to measure biochemical or physiological process in vivo. The first feature of PET is its ability to accurately measure the actual 3-D radiotracer distribution, which makes PET similar to autoradiography. The second feature is its ability to rapidly acquire a dynamic set of tomographic images through a volume of tissue. This is unique for PET imaging because no other imaging modality except MRI. The third feature of PET is the ability to acquire whole body images. It is the combination of these three features with the high specificity of receptor binding of biomolecules that makes PET imaging using radiolabeled biomolecules extremely attractive for nuclear medicine.

^{94m}Tc is a cyclotron-produced isotope with a half-life of 52 min (0.9 h) and the β⁺ energy of 2.47 MeV (72%). It can be obtained from a number of production methods, including ⁹⁴Mo(p, n)/^{94m}Tc (13.5-11 MeV), ^{nat}Nb(³He, 2n)/^{94m}Tc (18-10 MeV), ⁹²Mo(α, pn)/^{94m}Tc (26-18 MeV). The quantitative superiority of PET permits modeling of radiotracer kinetics and dosimetry measurements. The successful preparation of ^{94m}Tc in the pertechnetate form allows the use of the same kit for ^{99m}Tc radiopharmaceuticals to prepare ^{94m}Tc analogs. The use of dual isotopes ^{99m}Tc/^{94m}Tc (SPECT/PET) may provide much better imaging quality of diseased tissue. The integration of PET and SPECT radiotracer development would pave the way for better exploitation of the strengths of the two imaging modalities, and is contemplated for both the oncology and cardiology applications of radiopharmaceuticals disclosed herein.

There are many Tc cores for routine synthesis of ^{99m}Tc radiopharmaceuticals. The [Tc═O]³⁺ core is stable in the presence of a strong chelator in aqueous media. It is the most frequently used Tc core for ^{99m}Tc radiopharmaceuticals. Without limiting the current invention to any particular mechanism, it is believed that the [Tc═O]³⁺ core forms square pyramidal Tc-oxo chelates with tetradentate chelators, including N₄ propylene amine oxime (PnAO), N₃S triamidethiols, N₂S₂ diamidedithiols (DADS), N₂S₂ monoamidemonoamine-dithiols (MAMA), and N₂S₂ diaminedithiols (DADT). The [Tc═N]²⁺ core is isoelectronic with the [Tc═O]³⁺core. The nitrido ligand is a powerful π-electron donor and shows a high capacity to stabilize the Tc(V) oxidation state. The [Tc═N]²⁺ core forms Tc(V) nitrido complexes with a variety of chelators. Various chelators have been used for preparation of ^{99m}Tc radiopharmaceuticals. ^{99m}Tc-labeling techniques have been extensively reviewed and the following references are hereby incorporated by reference Hom, R. K. and Katzenellenbogen, J. A. Nucl. Med. Biol. 1997, 24, 485; Dewanjee, M. K. Semin. Nucl. Med. 1990, 20, 5; Jurisson, et al Chem. Rev. 1993, 93, 1137; Dilworth, J. R. and Parrott, S. J. Chem Soc. Rev. 1998, 27, 43; Liu, et al Bioconj. Chem. 1997, 8, 621; Liu, et al Pure & Appl. Chem. 1991, 63, 427; Griffiths, et al Bioconj. Chem. 1992, 3, 91; Liu, et al Chem. Rev. 1999, 99, 2235.

PCT application WO 90/06137, hereby incorporated by reference, disclosed a series of technetium-nitrido chelates of dithiocarbamates, including dimethyldithiocarbamate, di-n-propyl dithiocarbamate, N-ethyl-N-(2-ethyoxyethyl)dithiocarbamate. PCT applications WO 89/08657 and WO 92/00982, and WO 93/01839, hereby incorporated by reference, disclose processes for producing technetium nitrido complexs, which comprises reacting a polyphosphine as a reducing agent for the technetium oxide, then reacting with a nitride salt of a metal or ammonium ion. Since Tc-nitrido core has four to five coordination sites for various ligands or chelators, the choice of chelator may affect solution stability and number of radioactive species formed during ligand exchange reactions.

U.S. Pat. Nos. 5,288,476 and 6,071,492, hereby incorporated by reference, disclose cardiac tropism radiopharmaceutical products incorporating a nitride complex of transition metal and having a significant myocardial clearance.

U.S. Pat. Nos. 6,329,513 and 5,589,576, hereby incorporated by reference, disclose ^{99m}Tc complexes which comprises the moiety Tc═NR, Tc—N═NY or Tc—(N═N—Y)₂, and a synthetic organic ligand which confers biological target-seeking properties on the Tc complex.

Macrocyclic crown ethers have been the subject of intensive research for their capability to bind metal ion such as K⁺ and Na⁺. The extracellular Na⁺ concentration is 133-145 mM as compared to 3.5-4.8 mM for K⁺. However, the cytosolic Na⁺ concentration is only 10-40 mM as compared to 120 mM (upper limit) for K⁺. Although the 12- and 15-membered crown ether may not be able to form stable K⁺ complexes, the 18-membered crown ether group may result in a stronger interaction with K⁺. Therefore, the K⁺ binding capability may serve as a driving force for accumulation and retention of ^{99m}Tc complexes in myocardium; however, the applicant does not intend their invention be limited by any particular mechanism.

The technetium and rhenium radionuclides are preferably in their chemical form of [^{99m}Tc]pertechnetate or [^{186/188}Re]perrhenate and a pharmaceutically acceptable cation. The [^{99m}Tc]pertechnetate salt form is preferably sodium [^{99m}Tc]pertechnetate such as obtained from commercial ^{99m}Tc generators. The amount of [^{99m}Tc]pertechnetate used to prepare the metal complexes of the present invention can range from 1 mCi to 1000 mCi, or more preferably from 1 mCi to 50 mCi. Since the applicant is not aware of effective chemistry that can be used to attach the [^{99m}Tc]pertechnetate anion to an organic chelator, the [^{99m}Tc]pertechnetate is reduced by a reducing agent to a lower oxidation state in order to produce a stable ^{99m}Tc complex or to a reactive intermediate complex from which ^{99m}Tc can be easily transferred to the new chelator to form the expected ^{99m}Tc complex The rhenium chemistry is very similar to technetium chemistry due to the periodic relationship. Therefore, the methods used for molecules labeled with ^{99m}Tc should apply to those labeled with ^{186/188}Re.

Suitable reducing agents for the synthesis of radiopharmaceuticals of the present invention include stannous salts, dithionite or bisulfite salts, borohydride salts, and formamidinesulfinic acid, wherein the salts are of any pharmaceutically acceptable form. The preferred reducing agent is a stannous salt. The amount of a reducing agent used can range from 0.001 mg to 10 mg, or more preferably from 0.005 mg to 1 mg.

The total time of preparation will vary depending on the chemical properties of the metallic radionuclide, the identities and amounts of the reactants and the procedure used for the preparation. The preparations may be complete, resulting in >80% yield of the metal complex, in 1 minute or may require more time. After the radiolabeling, the resulting reaction mixture may optionally be purified using one or more chromatographic methods, such as Sep-Pack or high performance liquid chromatography (HPLC). The preferred methods are those, in which the ^{99m}Tc complex is prepared in high yield and high radiochemical purity without post-labeling purification.

The amounts of the ligand and coligand used for preparation of radiometal chelates can range from 1 mg to 1000 mg, or more preferably from 1 mg to 10 mg. One skilled in the art will be able to identify that the exact amount of the ligand and coligand needed is a function of the identity of a specific metal chelate, the procedure used for preparation of the metal chelate, and the amount and identities of the reactants used for the radiolabeling.

Another aspect of the present invention is a diagnostic kit for preparation of cationic metal complex radiopharmaceuticals useful as imaging agents for the diagnosis of cardiovascular disorders, infectious disease, inflammatory disease and cancer. Diagnostic kits of the present invention comprise one or more vials containing the sterile, non-pyrogenic, formulation comprised of a predetermined amount of the ligand described in this invention, a stabilizing coligand, a reducing agent, and optionally other components such as buffer agents, lyophilization aids, stabilization aids, solubilization aids and bacteriostats.

Another aspect of the present invention is related to the use of the said cationic ^{99m}Tc complexes as radiopharmaceuticals for diagnosis of cardiovascular disorders and cancer. For ^{99m}Tc complex radiopharmaceuticals, the biodistribution is exclusively determined by the physical properties of the metal complex. The use of ligating groups (for example, the combination of a crowned DTC chelator with a bisphosphine coligand) offers many opportunities to control the physical and biological characteristics of the cationic radiometal complex. The extent of such control is dependent on the choice of ligating groups, and the degree of functionalization of both the crowned DTC chelator and the bisphosphine coligand.

Another aspect of this invention is further related to methods of preparing said cationic ternary ligand ^{99m}Tc complex radiopharmaceuticals.

Another aspect of this invention is further related to radiopharmaceutical compositions comprising cationic ternary ligand ^{99m}Tc complexes.

Another aspect of this invention is further related to the cationic ternary ligand ^{186/188}Re complexes as radiopharmaceuticals for radiotherapy of cardiovascular disorders and cancer. Rhenium shares the similar coordination chemistry with technetium due to their periodic relationship. Rhenium has two isotopes (186Re and ¹⁸⁸Re) that might be useful for radiotherapy. ¹⁸⁶Re has a half-life of 3.68 days with a β-emission (Emax=1.07 MeV, 91% abundance) and a gamma-photon (E=137 keV, 9% abundance) which should allow imaging during therapy. ¹⁸⁶Re is a reactor-produced radionuclide and is obtained by the irradiation of ¹⁸⁵Re with neutrons (¹⁸⁵Re(n, γ)¹⁸⁶Re). The yield of ¹⁸⁶Re depends on the amount of Re target, the energy of the neutrons available, and the neutron reflux. The specific activity is low or medium, but a carrier-free product is not possible.

¹⁸⁸Re has a half-life of 16.98 h with a high-energy β-emission (Emax=2.12 MeV, 85% abundance) and 155 keV gamma photons (15% abundance). ¹⁸⁸Re can be prepared either from the nuclear reaction (¹⁸⁷Re(n, γ)¹⁸⁸Re) or from the ¹⁸⁸W-¹⁸⁶Re generator. The generator-produced ¹⁸⁸Re is carrier-free and has very high specific activity. The major advantage of using ¹⁸⁸Re in therapeutic nuclear medicine is the inexpensive and readily available ¹⁸⁸W-¹⁸⁶Re generator, which has a very long useful shelf-life.

In addition to the cardiology applications, cationic ^{99m}Tc complexes described invention can also be used as radiopharmaceuticals for non-invasive imaging of tumors—and tumor MDR1 (multidrug resistance) p-glycoprotein (Pgp) transport function—Various cationic ^{99m}Tc complex radiopharmaceuticals, originally developed for myocardial perfusion imaging, have been shown to be substrates for transport by MDR1Pgp.

Other features of the invention will become apparent in the course of the following descriptions of exemplary embodiments which are given for illustration of the invention and are not intended to be limiting thereof.

### EXAMPLES

Instruments. Chemicals and reagents were purchase from Sigma/Aldrich (St Louis). ¹H NMR spectra were recorded on a 300 MHz Bruker spectrometer. The ¹H NMR data were reported as δ in ppm relative to TMS. The radio-HPLC methods used a LabAlliance semi-prep HPLC system with a UV/visible detector (λ=230 nm), a β-ram IN-US radio-detector, and a Zorbax C₈ column (4.1 mm×150 mm, 100 Å pore size).

### General Procedure for Preparation of N-Substituted Bis(2-Diethoxyphosphorylethyl)-Amine (1)

A mixture of the amine (2.0 mmol) and diethyl vinylphosphonate (4.4-6.0 mmol) in 10 ml of ethanol was refluxed for 7-12 days. The solvent was evaporated and the crude product was purified by column chromatography using silica gel or neutral alumina as the solid phase and a mixture of CH₂Cl₂-methanol (20:1=v:v) as the mobile phase.

### N-Methoxyethyl-N,N-Bis(2-diethoxyphosphorylethyl)amine (1a)

The yield was 77%. ¹H NMR (CDCl₃, chemical shift δ in ppm relative to TMS): 4.09 (m, OCH₂CH₃, 8H), 3.44 (t, J=5.7 Hz, CH₃OCH₂, 2H), 3.33 (s, CH₃OCH₂, 3H), 2.82 (dt, J=7.7, 8.1 Hz, NCH₂CH₂P, 4H), 2.64 (t, J=5.7 Hz, OCH₂CH₂, 2H), 1.92 (m, NCH₂CH₂P, 4H), 1.32 (t, 7.0 Hz, OCH₂CH₃, 12H). ¹³C NMR (CDCl₃, chemical shift δ in ppm relative to TMS): 70.7, 61.4 (d, J=6.2 Hz), 58.8, 52.2, 46.9, 23.1 (d, J=136.4 Hz), 16.3 (d, J=5.8 Hz). ³¹P NMR (CDCl₃, chemical shift δ in ppm relative to 85% phosphoric acid): 28.6 ppm.

N-Ethoxyethyl-N,N-Bis(2-diethoxyplosphorylethyl) amine (1b). Column chromatography was run on silica gel. The yield was 74%. ¹H NMR (CDCl₃, chemical shift in ppm relative to TMS): 4.08 (m, OCH₂CH₃, 8H), 3.49 (t, J=5.7 Hz, CH₃CH₂OCH₂, 21H), 3.47 (q, J=7.0 Hz, CH₃CH₂OCH₂, 2H), 2.81 (dt, J=7.7, 8.1 Hz, NCH₂CH₂P, 4H), 2.64 (t, J=5.7 Hz, OCH₂CH₂, 2), 1.93 (m, NCH₂CH₂P, 4H), 1.32 (t, J=7.0 Hz, OCH₂CH₃, 12H), 1.18 (t, J=7.0 Hz, CH₃CH₂OCH₂, 3H). ¹³C NMR (CDCl₃, chemical shift δ in ppm relative to TMS): 68.7, 66.4, 61.4 (d, J=6.2 Hz), 52.3, 47.1, 23.3 (d, J=136.3 Hz), 16.4 (d, J=5.8 Hz), 15.1. ³¹P NMR (CDCl₃, chemical shift δ in ppm relative to 85% phosphoric acid): 28.7 ppm.

N-Benzyl-N,N-Bis(2-diethoxyphosphorylethyl)amine (1c). Column chromatography was run on silica gel. The yield was 94%. ¹H NMR (CDCl₃, chemical shift in ppm relative to TMS): 7.28 (m, C₆H₅ 5H), 4.03 (m, OCH₂CH₃, 8H), 3.56 (s, PhCH₂, 2H), 2.77 (dt, J=7.7, 8.1 Hz, NCH₂CH₂P, 4H), 1.93 (m, NCH₂CH₂P, 4H), 1.29 (t, J=7.0 Hz, OCH₂CH₃, 12H). ³¹P NMR (CDCl₃, chemical shift δ in ppm relative to 85% phosphoric acid): 28.6 ppm.

N-[(15-crown-5)-2-yl]methyl-N,N-Bis(2-diethoxyphosphorylethyl)amine (1d). Column chromatography was run on neutral alumina. The yield was 67%. ¹H NMR (CDCl₃, chemical shift δ in ppm relative to TMS): 4.09 (m, OCH₂CH₃, 8H), 3.78-3.55 (m, crown ether group, 19H), 2.82 (dt, J=7.5, 7.8 Hz, NCH₂CH₂P, 4H, 2.51 (d, J=4.5 Hz, OCHCH₂N, 2H), 1.92 (m, NCH₂CH₂P, 4H), 1.31 (t, J=7.0 Hz, OCH₂CH₃, 12H). ¹³C NMR (CDCl₃, chemical shift δ in ppm relative to TMS): 78.2, 71.9, 70.9 (br s), 70.8, 70.4 (br s), 70.0 (crown ether carbons), 61.4 (d, J=62 Hz), 54.4, 23.4 (d, J=136.3 Hz), 16.4 (d, J=5.8 HZ). ³¹P NMR (CDCl₃, chemical shift δ in ppm relative to 85% phosphoric acid): 28.7 ppm.

N-[(18-crown-6)-2-yl]methyl-N,N-Bis(2-Diethoxyphosphorytethylamie (1e). Refluxed for 11 days. Column chromatography was run on neutral alumina. Yield: 75%. ¹H NMR (CDCl₃, chemical shift δ in ppm relative to TMS): 4.09 (m, OCH₂CH₃, 8H), 3.78-3.55 (m, crown ether group, 23H), 2.78 (dt, J=7.5, 7.8 Hz, NCH₂CH₂P, 4H), 2.50 (d, J=4.5 Hz, OCHCH₂N, 2H), 1.90 (m, NCH₂CH₂P, 4H), 1.31 (t, J=7.0 Hz, OCH₂CH₃, 12H). ³¹P NMR (CDCl₃, chemical shift 5 in ppm relative to 85% phosphoric acid): 28.8 ppm.

### General Procedure for Preparation of N-Substituted Bis(2-Phosphinoethyl)amine (2)

All manipulations were strictly carried out under nitrogen atmosphere by using standard Schlenk line. A solution of the bisphosphonate 1 (2.0 mmol) in 5 mL of anhydrous THF was dropwise added into a stirred suspension of lithium aluminum hydride (8.0 mmol) in 5 mL of THF. After the initial exothermal reaction subsided, the reaction mixture was heated under reflux for 16 h. The reaction mixture was cooled to room temperature, and 20 mL of ether was added into it. The excess lithium aluminum hydride was hydrolyzed by cautious addition of 3% sodium hydroxide solution (2-3 mL). The ethereal layer was separated from the precipitate via cannula transfer. Another 20 mL of ether was used to wash the precipitate. The combined ether layers were dried over sodium sulfate, then filtered, and evaporated to afford the desired product 2 as colorless liquid. This crude product was used in next step reaction without further purification and characterization.

### General Procedure for Preparation of N-Substituted N,N-Bis[2-(Bis(3-Ethoxypropyl)phosphino)ethyl]amine (3)

All manipulations were strictly carried out under nitrogen atmosphere by using standard Schlenk line A mixture of bisphosphine 2 (1.0 mmol), allylethyl ether (9.0 mmol), and VAZO 67 (1,1′-azobis(cyclohexanecarbonitrile), 0.2 mmol) in 5 mL of ethanol was heated to reflux for 20 h. The solvent along with excess allylethyl ether was removed under vacuum and the residue was mixed thoroughly with 3 mL of 6 M hydrochloric acid. The resulting mixture was extracted twice with diethyl ether (2×10 mL) and the ethereal layer was discarded via cannula transfer. The pH of aqueous layer was adjusted >12 using 20% (w/w) sodium hydroxide solution. The aqueous layer was extracted 3 times with ether (3×10 mL). The combined ether layers were dried over sodium sulfate, and filtered, and then acidified with stirring by adding 4 M hydrogen chloride in dioxane until there was no more white precipitate. The supernant solution was separated from the precipitate and discarded. The precipitate was then washed twice with diethyl ether (2×10 mL), and dried under vacuum to afford the desired product 3 as colorless viscous oil. Since all bisphosphines are extremely air-sensitive under basic conditions, they were all isolated as the HCl salt forms, and should be stored under inert atmosphere.

### N-Methoxyethyl-N,N-Bis[2-(Bis(3-Ethoxypropyl)phosphino)ethyl]amine (3a)

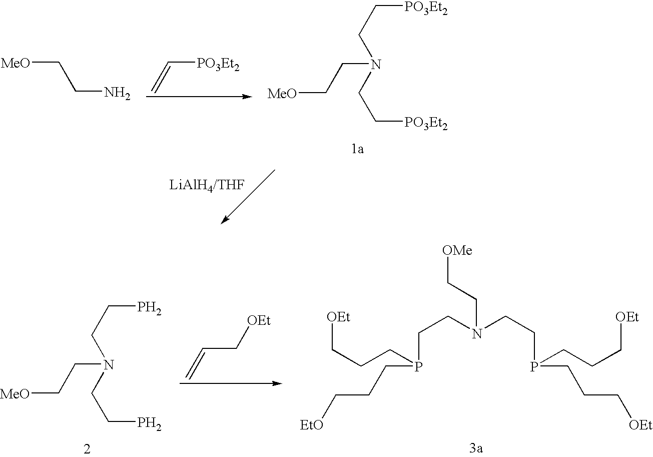

The yield was 68%. ¹H NMR (CDCl₃, chemical shift δ in ppm relative to TMS): 4.00-3.20 (m, CH₃OCH₂CH₂N, NCH₂CH₂P, CH₂OCH₃, 28H), 3.38 (s, CH₃OCH₂, 3H), 2.72 (m, PCH₂CH₂CH₂, 8H), 2.01 (m, PCH₂CH₂CH₂, 8H), 1.20 (t, J=7.0 Hz, OCH₂CH₃, 12H). ³¹P NMR (CDCl₃, chemical shift δ in ppm relative to 85% phosphoric acid): 12.0 ppm ESI-MS: m/z=539 (M+H).

N-Ethoxyethyl-N,N-Bis[2-(Bis(3Ethoxypropyl)phosphino)ethyl]amine (3b): The yield was 74%. ¹H NMR (CDCl₃, chemical shift δ in ppm relative to TMS): 4.10-3.20 (m, CH₃CH₂OCH₂CH₂N, NCH₂CH₂P, CH₂OCH₂CH₃, 30H), 2.62 (m, PCH₂CH₂CH₂, 8), 2.05 (m, PCH₂CH₂CH₂, 8H), 1.20 (t, J₁=6.9 Hz, OCH₂CH₃, 12H), 1.18 (t, J=7.0 Hz, CH₃CH₂OCH₂CH₂N, 3H). ³¹P NMR(CDCl₃, chemical shift δ in ppm relative to 85% phosphoric acid): 11.5 ppm. ESI-M: m/z=553 (M+H).

N-Benzyl-N,N-Bis[2-(Bis(3-Ethoxypropyl)phosphino)ethyl]amine (3c): The yield was 81%. ¹H.NMR (CDCl₃, chemical shift δ in ppm relative to TMS): 7.97-7.41 (m, phenyl, 5H), 4.66 (s, PCH₂, 2H), 4.00-3.20 (m, NCH₂CH₂P, CH₂OCH₂CH₃, 24H), 2.62 (m, PCH₂CH₂CH₂, 8H), 1.94 (m, PCH₂CH₂CH₂, 81), 1.16 (t, J=6.9 Hz, OCH₂CH₃, 12H). ³¹P NMR (CDCl₃, chemical shift δ in ppm relative to 85% phosphoric acid): 11.5 ppm. ESI-MS: m/z=572 (M+H).

N-[15-Crown-5)-2-yl]-N,N-Bis[2-(Bis(3-Ethoxypropyl)phosphino)ethyl]amine (3d): The yield was 61%. ¹H NMR (CDCl₃, chemical shift δ ppm relative to TMS): 4.10-3.20 (m, crown ether group, NCH₂CH₂P, CH₂OCH₂CH₃, 45H), 2.70 (m, PCH₂CH₂CH₂, 8H), 2.03 (m, PCH₂CH₂CH₂, 8H), 1.20 (t, J=7.0 Hz, OCH₂CH₃, 12H). ³¹P NMR (CDCl₃, δ in ppm relative to 85% phosphoric acid): 11.4 ppm. ESI-MS: m/z=714 (M+H).

N-[18-Crown 2-yl]N,N-Bis[2-(Bis(3-Ethoxypropyl)phosphino)ethyl]amine (3e): The yield was 65%. ¹H NMR (CDCl₃, chemical shift δ in ppm relative to TMS): 4.10-3.20 (m, crown ether group, NCH₂CH₂P, CH₂CH₂CH₃, 49H), 2.69 (m, PCH₂CH₂CH₂, 8H), 2.03 (m, PCH₂CH₂CH₂, 8H), 1.19 (t, J=7.0 Hz, OCH₂CH₃, 12H). ³¹P NMR (CDCl₃, δ in ppm relative to 85% phosphoric acid): 11.4 ppm. ESI-MS: m/z=758 (M+H).

N-Methoxyethyl-N,N-Bis[2-Bis(3-Methoxypropyl)phosphino)ethyl]amine (3f). A mixture of bisphosphine 2 (1.0 mmol), allylmethyl ether (9.0 mmol), and VAZO 67 (2,2′-azobis(2-methylbutyronitrile)) (0.2 mmol) in 5 mL of ethanol was added into a 50 ml, Schlenk tube equipped with Teflon stopcock. The tube was sealed and immersed into an 80° C. oil bath. The reaction mixture was stirred for 20 h. After cooled to room temperature, the reaction was worked up as described in general procedure. The yield was 95%. ¹H NMR (CDCl₃, chemical shift in ppm relative to TMS): 4.10-3.20 (m, CH₃OCH₂CH₂N, NCH₂CH₂P, CH₂OCH₃, 2H), 3.38 (s, CH₃OCH₂, 3H), 3.35 (s, PCH₂CH₂OCH₃, 12H, 2.69 (m, PCH₂CH₂CH₂, 8H), 2.02 (m, PCH₂CH₂CH₂, 8H). ³¹P NMR (CDCl₃, chemical shift in ppm relative to 85% phosphoric acid): 11.4 ppm. ESI-M: m/z=483 (M+H).

N-Ethoxyethyl-N,N-Bis[2-Bis(3-Methoxypropyl)phosphino)ethyl]amine (3 g): The yield was 84%. ¹H NMR (CDCl₃, chemical shift 5 in ppm relative to TMS): 4.10-3.20 (m, CH₃CH₂OCH₂CH₂N, NCH₂CH₂P, CH₂OCH₃, 22H), 3.35 (s, PCH₂CH₂CH₂OCH₃, 12H), 2.63 (m, PCH₂CH₂CH₂, 8H), 1.99 (m, PCH₂CH₂CH₂, 8H), 1.18 (t, J=7.0 Hz, CH₃CH₂OCH₂CH₂N, 3H). ³¹P NMR (CDCl₃, chemical shift δ in ppm relative to 85% phosphoric acid): 10.4 ppm. ESI-M: m/z=497 (M+H).

Synthesis of Crowned DTCs.

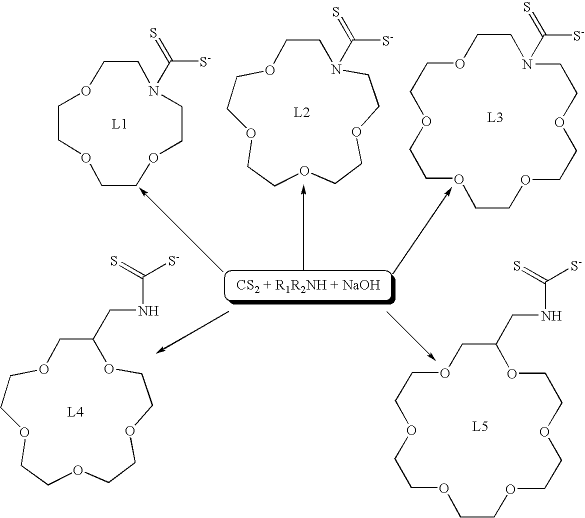

Crowned DTCs (L1-L5) were synthesized according to the Scheme above. Aza-crown ethers (1-aza-12-crown-4,1-aza-15-crown-5,1-aza-18-crown-6) and aminomethyl-crown ethers (2-aminomethyl-15-crown-5 and 2-aminomethyl-18-crown-6) are commercially available from Aldrich. The aza-crown or aminomethyl-crown ether reacts with carbon disulfide in the presence of sodium or potassium hydroxide to give the corresponding crowned DTC as its sodium or potassium salt in high yield (80-90%). L1-L5 were purified by recrystallization from a mixture of ethanol and diethyl ether.

### General Procedure for the Synthesis of Cationic ^{99m}Tc-Nitrido Complexes.

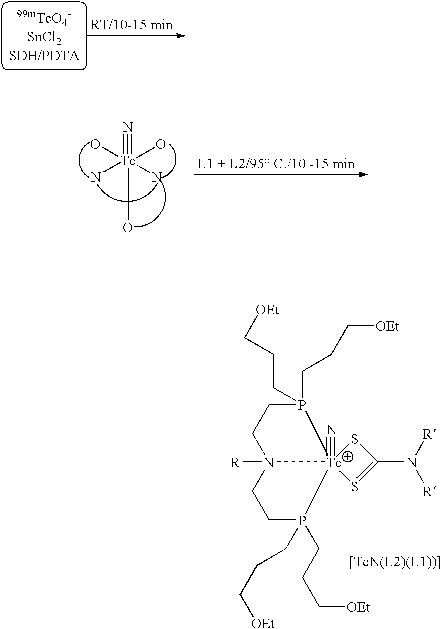

The solution containing succinic dihydrazide (SDH) and propylenediaminetetraacetic acid (PDTA) was prepared according to the literature procedure (Zhang, J. et al. J. Labelled Compounds & Radiopharm. 2000, 43: 693-700). To a 5 mL vial was added the solution containing PDTA (5 mg/mL) and SDH (5 mg/mL), followed by addition of 0.5-1.0 mL saline solution containing 2-10 mCi of ^{99m}TcO₄⁻. 10-30 μL SnCl₂ solution in 1.0 N HCl. The reaction mixture was kept at room temperature for 10-15 min to give the ^{99m}Tc-nitrido intermediate. After addition of 0.5 mL of solution containing sodium salt of the crown-ether containing dithiocarbamate (10 mg/mL) and bisphosphine coligand (10 mg/mL), the reaction mixture was heated at 95-100° C. for 10-15 min. The radiochemical purity (RCP) was evaluated by radio-HPLC. Table 1 summarizes the RCP data and radio-HPLC retention times of the synthesized cationic ^{99m}Tc-nitrido complexes.

### General Procedure for the Synthesis of Cationic ^{99m}Tc-Diazenido Complexes.

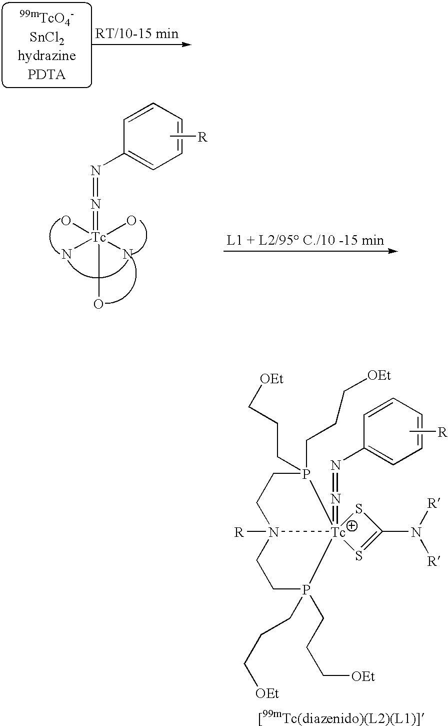

To a 5 mL vial was added the solution containing PDTA (4.5-5.0 mg/mL) and the aromatic hydrazine (1.0 mg/mL) dissolved in 0.5 mL of 0.5 M ammonium acetate, followed by the addition of 0.1-1.0 mL saline solution containing 2-10 mCi of ^{99m}TcO₄⁻, 10-30 μL. SnCl₂ solution in 1.0 N HCl. The reaction mixture was heated at 95-100° C. for 10-15 min to give the ^{99m}Tc-diazenido intermediate. After addition of 0.5 mL of a solution containing sodium salt of the crown-ether containing dithiocarbamate (10 mg/mL) and bisphosphine coligand (10 mg/mL) dissolved in 30-50% ethanol, the reaction mixture was heated at 95-100° C. for another 10-15 min. The radiochemical purity (RCP) was evaluated by radio-HPLC. Table 2 summarizes the RCP data and radio-HPLC retention times of the synthesized cationic ^{99m}Tc-diazenido complexes.

TABLE 1
The RCP data and radio-HPLC retention times of cationic ^{99m}Tc-nitrido
complexes.

| | Radiochemical | HPLC Retention | HPLC |
|---|---|---|---|
| Compound | Purity (RCP, %) | Time (Min) | Gradient* |
| [^{99m}TcN(3a)(L1a)]⁺ | >90% | 15.2 | A |
| [^{99m}TcN(3a)(L1b)]⁺ | >90% | 14.2 | A |
| [^{99m}TcN(3a)(L1c)]⁺ | >90% | 14.9 | A |
| [^{99m}TcN(3a)(L1d)]⁺ | >95% | 16.5 | B |
| [^{99m}TcN(3a)(L1e)]⁺ | >98% | 14.8 | A |
| [^{99m}TcN(3b)(L1a)]⁺ | ~80% | 15.4 | A |
| [^{99m}TcN(3b)(L1b)]⁺ | 84% | 15.0 | A |
| [^{99m}TcN(3b)(L1c)]⁺ | 85% | 15.8 | A |
| [^{99m}TcN(3b)(L1d)]⁺ | >95% | 12.5 | A |
| [^{99m}TcN(3b)(L1e)]⁺ | >95% | 13.5 | A |
| [^{99m}TcN(3c)(L1d)]⁺ | 98% | 12.3 | C |
| [^{99m}TcN(3C)(L1e)]⁺ | 95% | 12.5 | C |
| [^{99m}TcN(3d)(L1a)]⁺ | ~90% | 15.0 | A |
| [^{99m}TcN(3d)(L1b)]⁺ | ~80% | 15.2 | A |
| [^{99m}TcN(3d)(L1c)]⁺ | ~85% | 15.5 | A |
| [^{99m}TcN(3d)(L1d)]⁺ | ~95% | 13.5 | A |
| [^{99m}TcN(3d)(L1e)]⁺ | >95% | 15.2 | A |
| [^{99m}TcN(3e)(L1d)]⁺ | ~95% | 16.0 | A |

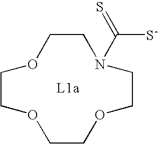

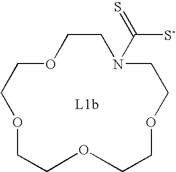

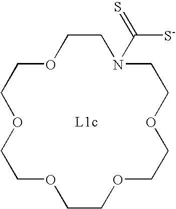

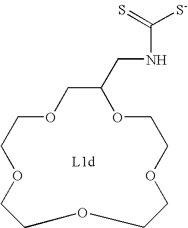

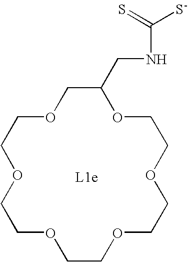

*The flow rate was 1 mL/min with a gradient mobile phase. Solvent A
was 10 mM ammonium acetate buffer (pH = 6.8), and solvent B (100%
methanol).

| | | | | | | |
|---|---|---|---|---|---|---|
| Gradient A, | Time (min): | 0 | 5 | 15 | 25 | 30 |
| | Solvent A (%): | 20 | 20 | 10 | 10 | 20 |
| | Solvent B (%): | 80 | 80 | 90 | 90 | 80 |
| Gradient B, | Time (min): | 0 | 5 | 15 | 15 | 30 |
| | Solvent A (%): | 30 | 20 | 10 | 30 | 20 |
| | Solvent B (%): | 70 | 80 | 90 | 70 | 80 |
| Gradient C, | Time (min): | 0 | 5 | 15 | 15 | 30 |
| | Solvent A (%): | 20 | 20 | 0 | 0 | 20 |
| | Solvent B (%): | 80 | 80 | 100 | 100 | 80 |

TABLE 2
The RCP data and radio-HPLC retention times of cationic
^{99m}Tc-diazenido complexes.

| | Radiochemical | HPLC | |
|---|---|---|---|
| | Purity | Retention | HPLC |
| Compound | (RCP, %) | Time (Min) | Gradient* |
| [^{99m}Tc(4-HO₂CPhN₂)(3a) | ~95% | 14.9 | B |
| (L1d)]⁺ | | | |
| [^{99m}Tc(4-PhN₂)(3a)(L1d)]⁺ | ~90% | 12.0 | B |
| [^{99m}Tc(4-MeOPhN₂)(3a) | ~90% | 14.8 | B |
| (L1d)]⁺ | | | |
| [^{99m}Tc(4-ClCPhN₂)(3a) | ~90% | 13.5 | B |
| (L1d)]⁺ | | | |
| [^{99m}Tc(PhN₂)(3a)(L1e)]⁺ | ~95% | 16.3 | B |
| [^{99m}Tc(4-ClPhN₂)(3a)(L1e)]⁺ | ~90% | 16.2 | B |
| [^{99m}Tc(4-ClPhN₂)(3c)(L1e)]⁺ | ~85% | 21.3 | B |
| [^{99m}Tc(4-MeOPhN₂)(3c) | ~95% | 20.5 | B |
| (L1e)]⁺ | | | |
| [^{99m}Tc(PhN₂)(3c)(L1e)]⁺ | ~85% | 21.2 | B |

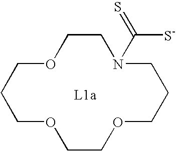

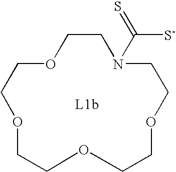

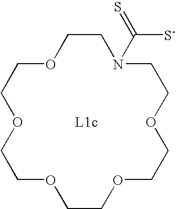

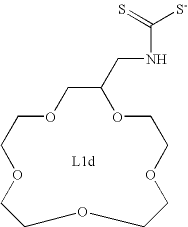

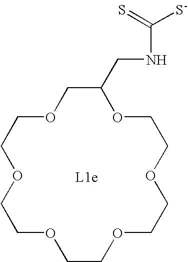

*The flow rate was 1 mL/min with a gradient mobile phase. Solvent A
was 10 mM ammonium acetate buffer (pH = 6.8), and solvent B (100%
methanol).

| | | | | | | |
|---|---|---|---|---|---|---|
| Gradient B, | Time (min): | 0 | 5 | 15 | 15 | 30 |
| | Solvent A (%): | 30 | 20 | 10 | 30 | 20 |
| | Solvent B (%): | 70 | 80 | 90 | 70 | 80 |

### Biodistribution Studies in the Rat Model

Animal studies will be performed following the literature procedures (Boschi, A. et al Nucl. Med. Commun 2002, 23: 689). Biodistribution studies were carried out using Sprague-Dawley rats in compliance NIH animal experiment guidelines (Principles of laboratory Animal Care, NIH Publication No. 86-23, revised 1985). These studies are designed as the preliminary screening tool to determine the biodistribution characteristics of cationic ^{99m}Tc complexes.

Sprague-Dawley rats (200-250 g) were anesthetized with an intramuscle injection of a mixture of ketamine (80 mg/kg) and xylazine (19 mg/g). They received the cationic ^{99m}Tc complex (1-10 μCi in 100 GL solution) via intravenous injection into the exposed jugular vein. The amount of activity administered to each animal was quantified, ultimately allowing the biodistribution of each radiopharmaceutical to be calculated as both a percentage of the injected dose per organ (% ID/organ) and a percentage of the injected dose per gram of tissue wet mass (% ID/g). The animals were sacrificed via exsanguinations and opening of thoracic cavity at selected time points; relevant tissues and organs were excised, weighed, and counted to determine the tissue uptake of the ^{99m}Tc complex. The organs of interest included heart, brain, blood, lung, liver, spleen, kidneys, muscle and intestines. Four rats were used at each selected time point to ensure acquisition of reliable biological data. Ideal ^{99m}Tc radiopharmaceuticals are those, which have high heart uptake, long heart retention time, and rapid blood clearance, preferably via renal system. This model can also be used to evaluate radiopharmaceuticals of the present invention comprised of a positron emitting isotope such as ^{94m}Tc.

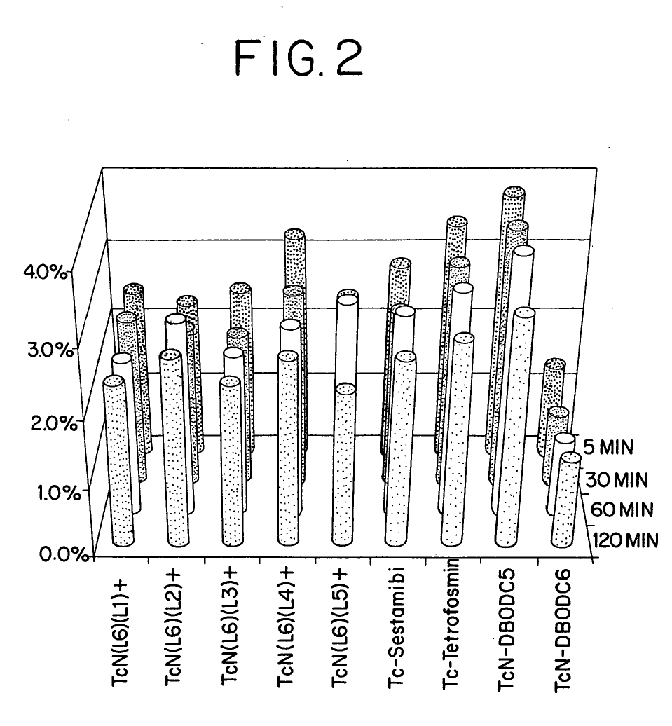

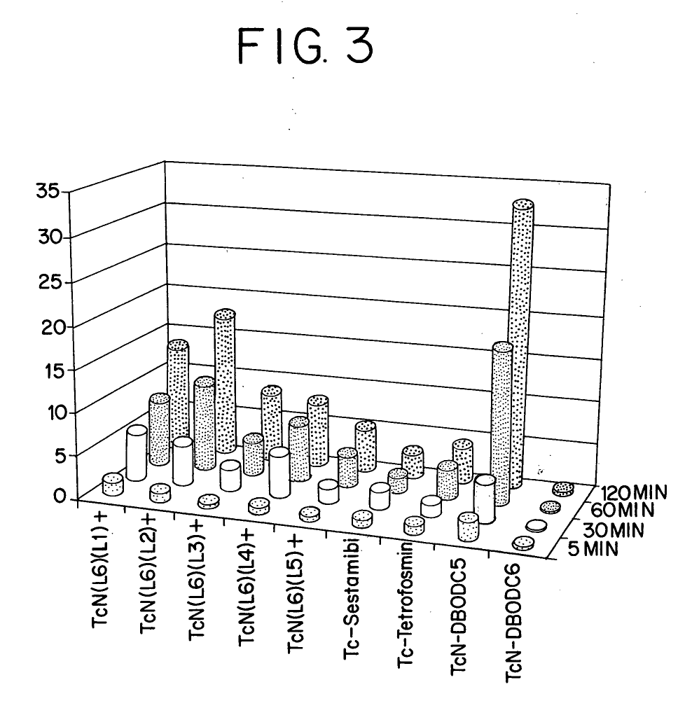

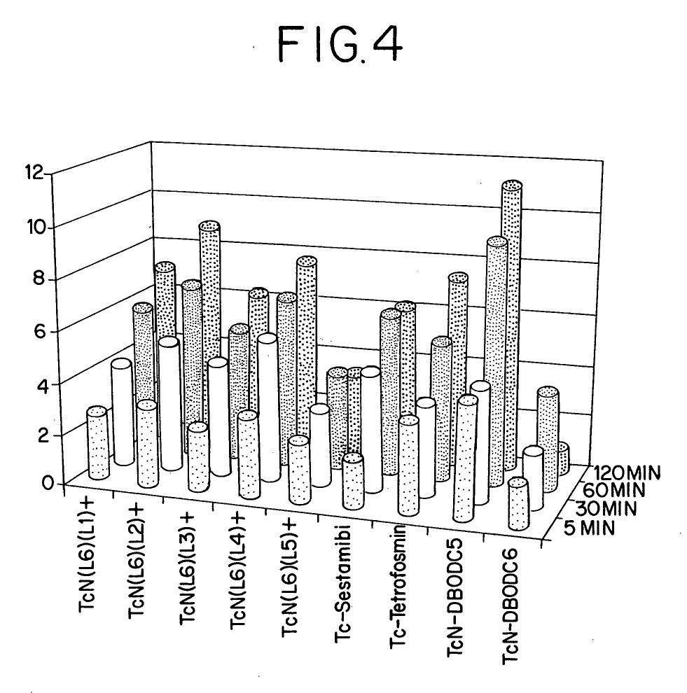

Tables 3-7 list the organ uptake expressed as the injected dose per gram of wet tissue mass (% ID/g) and T/B ratios for complexes [^{99m}TcN(3a)(DTC)]⁺ (DTC=L1a−L1e). FIG. 2 shows a direct comparison of heart uptake of complexes [^{99m}TcN(3a)(DTC)]⁺ (DTC=L1a−L1e) with known heart imaging agents (^{99m}Tc-Sestamibi, ^{99m}Tc-Tetrofosmin, ^{99m}TcN-DBODC5, and ^{99m}TcN-DBODC6) in the same animal model. FIG. 3 shows the direct comparison of heart/liver ratios of complexes [^{99m}TcN(3a)(DTC)]⁺ (DTC=L1a−L1e), ^{99m}Tc-Sestamibi, ^{99m}Tc-Tetrofosmin, ^{99m}TcN-DBODC5, and ^{99m}TcN-DBODC6 at 30 min, 60 min and 120 min postinjection. FIG. 4 shows the direct comparison of heart/lung ratios of cationic complexes [^{99m}TcN(3a)(DTC)]⁺ (DTC=L1a−L1e), ^{99m}Tc-Sestamibi ^{99m}Tc-Tetrofosmin, ^{99m}TcN-DBODC5, and ^{99m}TcN-DBODC6 at 30 min 60 min and 120 min postinjection. The heart uptake and T/B ratios for ^{99m}TcN-DBODC5, ⁹⁹ⁿTcN-DBODC6, ^{99m}Tc-Sestamibi, and ^{99m}Tc-Tetrofosmin were obtained from literature (Boschi, A. et al Nucl. Med. Commun. 2002, 23, 689; Boschi, A. et al. J. Nucl. Med. 2003, 44: 806-814).

In general, all five cationic complexes [^{99m}TcN(3a)(DTC)]⁺ (DTC=L1a−L1e) show a high initial heart uptake (2.31-3.29% ID/g at 5 min postinjection). They were also able to retain radioactivity in rat myocardium for >2 h (2.28-2.74% ID/g at 120 min postinjection). There is no significant washout from heart over 2 h. They also show a rapid clearance from blood, muscle, liver and lungs, which result in fairly high T/B ratios. Changing the crown ether group has a significant impact on both heart uptake and T/B ratios of cationic ^{99m}Tc-nitrido complexes. For example, the heart uptake of [^{99m}TcN(3a)(L1d)]⁺ was 3.29±0.32% ID/g at 5 min postinjection while the heart uptake of [^{99m}TcN(3a)(L1e)]⁺ was only 2.39±0.33% ID/g at the same time point. The complex [^{99m}TcN(3a)(L1d)]⁺ shows the highest heart uptake (329±0.32% ID/g at 5 min postinjection); but there is no significant difference in heart uptake of complexes ^{99m}TcN(3a)(DTC)⁺ (DTC=L1a−L1e) within the experimental error at latter time points. The complex [^{99m}TcN(3a)(L1b)]⁺ shows the fastest clearance from non-target organs with the best heart/liver (18:1) and heart/muscle (14:1) ratios at 120 min postinjection.

All five cationic complexes [^{99m}TN(3a)(DTC)]⁺ (DTC=L1a−L1e) show a higher heart uptake than that of ^{99m}TcN-DBODC6, reported by Duatti and coworkers (Boschi, A. et al Nucl. Med. Commun. 2002, 23, 689; Boschi A. et al. J. Nucl. Med. 2003, 44: 806-814). For example, the heart uptake of [^{99m}TcN(3a)(L1d)]⁺ is more than twice of ^{99m}TcN-DBODC6 during the 2 h study period. More importantly, the heart/liver ratio of [^{99m}TcN(3a)(L1b)]⁺ is ˜40 times better that of ^{99m}TcN-DBODC6 at 120 min postinjection and the heart/liver ratio of [^{99m}TcN(3a)(L1d)]⁺ is ˜20 times better that of ^{99m}TcN-DBODC6 between 60 min and 120 min postinjection Since L6 is almost identical to PNP6, it is quite clear that the crown ether groups significantly improve both the heart uptake and radioactivity clearance of complexes [^{99m}TcN(3a)(DTC)]⁺ (DTC=L1a−L1e) from non-target organs, such as blood, muscle, liver and lungs.

The heart/liver ratios of complexes [^{99m}TcN(3a)(DTC)]⁺ (DTC=L1a−L1e) are much better than those of both ^{99m}Tc-Sestamibi and ^{99m}Tc-Tetrofosmin. For example, the heart/liver ratio of the complex [^{99m}TcN(3a)(L1d)]⁺ is ˜4 times better that of ^{99m}Tc-Sestamibi and about twice of that of ^{99m}Tc-Tetrofosmin over 2 h. The heart/liver ratio of [^{99m}ScN(3a)(L1b)]⁺ is ˜8 times better than that of ⁹⁹Tc-Sestamibi and ˜4 times of that of ^{99m}Tc-Tetrofosmin at 120 min postinjection.

### General Protocol for Biodistribution Studies in Guinea Pigs

Animal studies can be performed following the literature procedure (Lisic, E. C., Heeg, M. J., and Deutsch, E. Nucl. Med. Biol. 1999, 26: 563-571; Marmion, M. E. et al. Nucl. Med. Biol. 1999, 26: 755-777.). Male Harley guinea pigs (400-600 g) are induced by IM injection of Ketamine (100 mg/kg) and Xylazine (20 mg/kg). After induction, the animal receives no more than 2 additional half dose injections to maintain a surgical plane of anesthesia. Depth of anesthesia will be monitored not less than every 15 min, checking for the heartbeat, respiration and pain response. Four guinea pigs per time point are used. Once the animal is in surgical plane of anesthesia, noted by lack of pain response, it is injected intravenously with 1-10 μCi of the cationic ^{99m}Tc complexes through a surgically exposed jugular vein. For imaging studies, animals are monitored on the gamma camera at the specified time (5 min, 60 min and 120 min postinjection) while animal are still under anesthesia. Upon completion of the study, images are evaluated by circumscribing the target region (heart) of interest (ROI) and a background site in the neck area below the carotid salivary glands. For biodistribution studies, four guinea pigs are euthanized by injection of Nembutal Sodium 50 mg/ml or Beuthanasia-D IP at a dose of 0.2 ml/100 g, opening of thoracic cavity, resulting irreversible death, and/or opening of thoracic cavity, resulting irreversible death, at the end of each selected time point (5, 60, 120 min postinjection). Organs of interest (blood, heart lung, liver, spleen, kidneys, muscle, and intestines) are removed and weighed immediately. Samples of animals injected with cationic ^{99m}Tc complexes are counted in a well-type gamma scintillation counter to determine the tissue distribution in different organs, and the mean total injected dose per gram (% ID/g) is calculated.

The diagnostic radiopharmaceuticals are administered by intravenous injection, usually in saline solution, at a dose of 1 to 100 mCi per 70 kg body weight, or preferably at a dose of 5 to 30 mCi. Imaging is performed using known procedures.

The therapeutic radiopharmaceuticals are administered by intravenous injection, usually in saline solution, at a dose of 0.1 to 100 mCi per 70 kg body weight, or preferably at a dose of 0.5 to 50 mCi per 70 kg body weight.

TABLE 3
Biodistribution data for [^{99m}TcN(3a)(L1a)]⁺ (% ID/gram of wet organ).

| Organ | 5 min | 30 min | 60 min | 120 min |
|---|---|---|---|---|
| Blood | 0.09 ± 0.02 | 0.03 ± 0.01 | 0.01 ± 0.00 | 0.01 ± 0.01 |
| Heart | 2.48 ± 0.36 | 2.48 ± 0.54 | 2.24 ± 0.68 | 2.38 ± 0.40 |
| Lungs | 0.88 ± 0.15 | 0.58 ± 0.10 | 0.42 ± 0.09 | 0.34 ± 0.05 |
| Liver | 1.22 ± 0.26 | 0.48 ± 0.24 | 0.29 ± 0.06 | 0.19 ± 0.04 |
| Kidneys | 9.40 ± 1.33 | 5.71 ± 1.53 | 4.28 ± 2.16 | 3.16 ± 0.82 |
| Intestine | 4.65 ± 1.23 | 8.20 ± 2.96 | 4.59 ± 2.10 | 1.59 ± 0.67 |
| Muscle | 0.19 ± 0.07 | 0.15 ± 0.02 | 0.18 ± 0.05 | 0.14 ± 0.03 |
| Heart/ | 26.8 ± 2.7 | 94.0 ± 25.7 | 132.9 ± 33.1 | 290.2 ± 87.0 |
| Blood | | | | |
| Ratio | | | | |
| Heart/ | 2.85 ± 0.25 | 4.17 ± 0.96 | 5.94 ± 0.70 | 7.13 ± 1.55 |
| Lung | | | | |
| Ratio | | | | |
| Heart/ | 2.01 ± 0.14 | 5.94 ± 1.28 | 8.20 ± 2.11 | 13.51 ± 3.40 |
| Liver | | | | |
| Ratio | | | | |
| Heart/ | 8.81 ± 1.44 | 9.67 ± 3.26 | 10.86 ± 3.24 | 14.09 ± 6.43 |
| Muscle | | | | |
| Ratio | | | | |

TABLE 4
Biodistribution data for [^{99m}TcN(3a)(L1b)]⁺ (% ID/gram of wet organ).

| Organ | 5 min | 30 min | 60 min | 120 min |
|---|---|---|---|---|
| Blood | 0.09 ± 0.01 | 0.03 ± 0.00 | 0.02 ± 0.00 | 0.01 ± 0.00 |
| Heart | 2.31 ± 0.60 | 2.34 ± 0.37 | 2.83 ± 0.82 | 2.74 ± 0.60 |
| Lungs | 0.68 ± 0.11 | 0.47 ± 0.02 | 0.40 ± 0.04 | 0.31 ± 0.06 |
| Liver | 1.89 ± 0.72 | 0.47 ± 0.12 | 0.27 ± 0.06 | 0.15 ± 0.03 |
| Kidneys | 7.90 ± 0.43 | 5.57 ± 0.89 | 4.63 ± 1.21 | 3.01 ± 0.35 |
| Intestine | 4.63 ± 0.96 | 5.79 ± 2.38 | 2.71 ± 1.03 | 1.10 ± 0.60 |
| Muscle | 0.23 ± 0.08 | 0.25 ± 0.02 | 0.27 ± 0.15 | 0.16 ± 0.02 |
| Heart/ | 25.4 ± 7.8 | 74.5 ± 15.8 | 105.3 ± 21.5 | 174.5 ± 72.9 |
| Blood | | | | |
| Heart/ | 3.36 ± 0.36 | 5.26 ± 0.90 | 7.06 ± 1.48 | 8.94 ± 3.10 |
| Lung | | | | |
| Heart/ | 1.47 ± 0.89 | 5.36 ± 2.00 | 10.97 ± 3.96 | 17.84 ± 6.69 |
| Liver | | | | |
| Heart/ | 1.47 ± 0.89 | 5.36 ± 2.00 | 10.97 ± 3.96 | 17.84 ± 6.69 |
| Muscle | | | | |

TABLE 5
Biodistribution data for [^{99m}TcN(3a)(L1c)]⁺ (% ID/gram of wet organ).

| Organ | 5 min | 30 min | 60 min | 120 min |
|---|---|---|---|---|
| Blood | 0.32 ± 0.08 | 0.13 ± 0.04 | 0.06 ± 0.01 | 0.03 ± 0.01 |
| Heart | 2.50 ± 0.75 | 2.21 ± 0.57 | 2.36 ± 0.75 | 2.35 ± 0.49 |
| Lungs | 1.04 ± 0.56 | 0.48 ± 0.03 | 0.47 ± 0.23 | 0.38 ± 0.06 |
| Liver | 3.08 ± 1.20 | 0.79 ± 0.32 | 0.60 ± 0.32 | 0.27 ± 0.04 |
| Kidneys | 12.33 ± 1.44 | 6.35 ± 1.29 | 5.63 ± 3.15 | 3.25 ± 0.59 |
| Intestine | 11.49 ± 9.67 | 3.69 ± 2.19 | 6.30 ± 8.43 | 1.71 ± 0.96 |
| Muscle | 0.38 ± 0.20 | 0.31 ± 0.08 | 0.23 ± 0.09 | 0.27 ± 0.05 |
| Heart/Blood Ratio | 7.71 ± 0.48 | 18.33 ± 9.58 | 37.00 ± 9.47 | 79.63 ± 18.15 |
| Heart/Lung Ratio | 2.58 ± 0.52 | 4.60 ± 1.02 | 5.35 ± 1.32 | 6.40 ± 2.01 |
| Heart/Liver Ratio | 0.83 ± 0.07 | 3.11 ± 1.47 | 4.48 ± 1.76 | 8.82 ± 1.02 |
| Heart/Muscle Ratio | 9.00 ± 2.35 | 7.59 ± 2.06 | 9.59 ± 1.80 | 8.87 ± 1.42 |

TABLE 6
Biodistribution data for [^{99m}TcN(3a)(L1d)]⁺ (% ID/gram of wet organ).

| Organ | 5 min | 30 min | 60 min | 120 min |
|---|---|---|---|---|
| Blood | 0.39 ± 0.05 | 0.06 ± 0.01 | 0.03 ± 0.00 | 0.03 ± 0.00 |
| Heart | 3.29 ± 0.32 | 2.84 ± 0.46 | 2.74 ± 0.70 | 2.73 ± 0.67 |
| Lungs | 1.11 ± 0.25 | 0.47 ± 0.05 | 0.42 ± 0.08 | 0.31 ± 0.07 |
| Liver | 3.17 ± 0.83 | 0.63 ± 0.10 | 0.43 ± 0.15 | 0.38 ± 0.07 |
| Kidneys | 12.35 ± 3.39 | 4.66 ± 1.05 | 3.55 ± 0.42 | 2.50 ± 0.21 |
| Intestine | 9.07 ± 5.68 | 9.75 ± 5.53 | 5.99 ± 4.19 | 4.48 ± 3.05 |
| Muscle | 0.37 ± 0.05 | 0.40 ± 0.20 | 0.36 ± 0.13 | 0.30 ± 0.05 |
| Heart/Blood Ratio | 8.06 ± 0.32 | 45.39 ± 5.27 | 82.17 ± 29.29 | 116.74 ± 46.32 |
| Heart/Lung Ratio | 3.24 ± 0.83 | 5.70 ± 1.16 | 6.85 ± 2.59 | 7.76 ± 0.99 |
| Heart/Liver Ratio | 1.08 ± 0.32 | 5.50 ± 1.65 | 7.20 ± 3.44 | 8.24 ± 2.28 |
| Heart/Muscle Ratio | 8.18 ± 0.79 | 7.27 ± 3.24 | 8.17 ± 3.03 | 8.13 ± 1.89 |

TABLE 7
Biodistribution data for [^{99m}TcN(3a)(L1e)]⁺ (% ID/gram of wet organ).

| Organ | 5 min | 30 min | 60 min | 120 min |
|---|---|---|---|---|
| Blood | 0.53 ± 0.04 | 0.13 ± 0.07 | 0.05 ± 0.01 | 0.02 ± 0.00 |
| Heart | 2.39 ± 0.33 | 2.51 ± 0.37 | 3.06 ± 0.33 | 2.28 ± 0.32 |
| Lungs | 0.94 ± 0.07 | 0.80 ± 0.21 | 0.79 ± 0.05 | 0.68 ± 0.10 |
| Liver | 3.60 ± 0.49 | 1.43 ± 0.73 | 0.83 ± 0.27 | 0.81 ± 0.29 |
| Kidneys | 9.09 ± 0.37 | 7.25 ± 2.38 | 5.88 ± 0.51 | 5.16 ± 0.57 |
| Intestine | 8.33 ± 1.15 | 11.15 ± 5.20 | 5.33 ± 2.95 | 2.11 ± 1.21 |
| Muscle | 0.34 ± 0.07 | 0.25 ± 0.08 | 0.37 ± 0.13 | 0.23 ± 0.05 |
| Heart/Blood Ratio | 4.50 ± 0.68 | 24.25 ± 9.24 | 69.95 ± 12.67 | 132.31 ± 10.21 |
| Heart/Lung Ratio | 2.55 ± 0.46 | 3.25 ± 0.48 | 3.89 ± 1.64 | 3.39 ± 1.39 |
| Heart/Liver Ratio | 0.67 ± 0.06 | 2.12 ± 0.97 | 4.07 ± 2.46 | 5.54 ± 2.50 |
| Heart/Muscle Ratio | 6.87 ± 1.01 | 6.76 ± 1.98 | 7.40 ± 1.63 | 7.54 ± 1.50 |

Although this invention has been described with respect to specific embodiments, the details of these embodiments are not to be construed as limitations. Various equivalents, changes and modifications may be made without departing from the spirit and scope of this invention, and it is understood that such equivalent embodiments are part of this invention.

We claim:

1. A novel crown ether-containing cationic metal complex radiopharmaceutical of the formula:
L1-MC-L2
and pharmaceutically acceptable salt thereof, wherein
MC is the metal core, and is selected from a group of [M≡N]²⁺, [M=N═N—R¹]²⁺, [M=O]³⁺, and [M=N—R²]³⁺;
M is the metallic radionuclide, and is selected from ^{99m}Tc, ^{94m}Tc, ¹⁸⁶Re and ¹⁸⁸Re;
R¹ and R² can be the same or different, and are independently selected, at each occurrence, from the group consisting of: C₁₋₁₀ alkyl substituted with 1-5 R³, and aryl substituted with 1-4 R⁴ and 0-1R⁵;
R³, R⁴ and R⁵ are independently selected, at each occurrence, from the group consisting of: H, F, Cl, Br, —OR⁶, CO₂R⁶, —OC(═O)R⁶, —OC(═O)OR⁶, —OCH₂CO₂R⁶, —NR⁷C(═O)OR⁶, —SO₂R⁶, —SO₃R⁶, —NR⁷SO₂R⁶, and —PO₃R⁶;
R⁶ and R⁷ can be the same or different, and are independently selected, at each occurrence, from the group comprising of: C₁₋₁₀ alkyl, aryl group, and macrocyclic crown ether-containing group;
L1 is a bidentate ligand with a combination of O, N, P, and S donor atoms; and
L2 is a tridentate coligand with a combination of phosphine-P, anine-N, and imine-N.

2. A crown ether-containing cationic metal complex radiopharmaceutical of claim 1, wherein:
MC is [M≡N]²⁺ or [M=N═N—R¹]²⁺, wherein:
M is ^{99m}Tc or ^{94m}Tc;
R¹ is selected from an aryl substituted with 1 or 2 R³; and
R³ is selected from the group consisting of: H, F, Cl, Br, —OR⁶, —CO₂R⁶, and —PO₃R⁶;
R⁶ is selected from the group comprising of: C₁₋₅ allyl and macrocyclic crown ether-containing group;
L1 is DTC chelator of the formula: 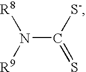
wherein R⁵ and R⁹ can be the same or different, and are independently selected, at each occurrence, from the group comprising of: H, C₁₋₁₀ alkyd C₃₋₁₀ alkoxyalkyl, aryl, and macrocyclic crown ether-containing group, or R¹ and R² may be taken together to form a macrocycle of the formula [(CH₂)ₐ—O]_{b}—(CH₂)_{c}, wherein
a is 2-5;
b is 3-8;
c is 2-5;
L2 is bisphosphine coligand of the formula: 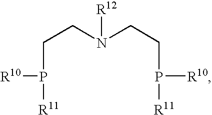
wherein R¹⁰ and R¹¹ can be the same or different, and are independently selected, at each occurrence, from the group comprising of: C₁₋₁₀ allyl and alkoxyalkyl;
R¹² is selected from the group comprising of: C₁₋₁₀ alkyl substituted with 1-5 R¹³ and a macrocyclic crown ether-containing group; and
R¹³ is selected the group consisting of: —OR¹⁴, —CO₂R¹⁴, CONR¹⁴R¹⁵, and —PO₃R¹⁴; and
R¹⁴ is R¹⁵ is selected from the group comprising of: C₁₋₁₀ alkyl.

3. A crown ether-containing cationic metal complex radiopharmaceutical of claim 2, wherein:
R¹ is selected from an aryl substituted with R³;
R³ is selected from the group consisting of: H, Cl, —OR⁶, and CO₂R⁶;
R⁶ is selected from methyl or ethyl group;
R⁸ and R⁹ can be the same or different, and are independently selected, at each occurrence, from the group comprising of: H, C₁₋₁₀ alkyl, C₃₋₅ alkoxyalkyl, and macrocyclic crown ether-containing group, or R¹ and R² may be taken together to form a macrocycle of the formula [(CH₂)ₐ—O]_{b}—(CH₂)_{c}, wherein
a is 2 or 3;
b is 3-6;
c is 2 or 3;
R¹⁰ and R¹¹ are alkoxyalkyl groups; and
R¹² is an alkoxyalkyl group or a macrocyclic crown ether-containing group.

4. A crown ether-containing cationic metal complex radiopharmaceutical of claim 3, wherein:
R⁸ and R⁹ are independently selected, at each occurrence, from the group comprising of:
H, C₃₋₅ alkoxyalkyl, and macrocyclic crown ether-containing group, or R¹ and R² may be taken together to form a macrocycle of the formula [(CH₂)ₐ—O]_{b}—(CH₂)_{c}, wherein
a is 2;
c is 2;
R¹⁰ and R¹¹ are selected from a group of: methoxypropyl, methoxyethyl, ethoxypropyl, and ethoxyethyl; and
R¹² is a macrocyclic crown ether-containing group.

5. A crown ether-containing cationic metal complex radiopharmaceutical of claim 1, wherein L1 is selected from any one of the following crown-ether containing chelator of the formula:

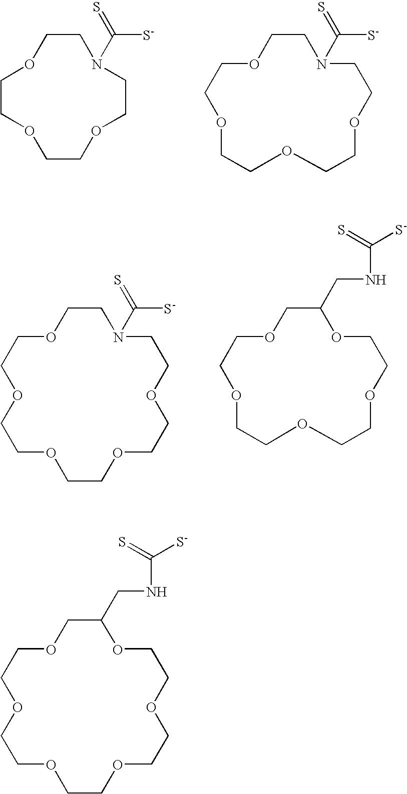

6. A crown ether-containing cationic metal complex radiopharmaceutical of claim 1, wherein L2 is selected from any one of the following bisphosphine coligands of the formula:

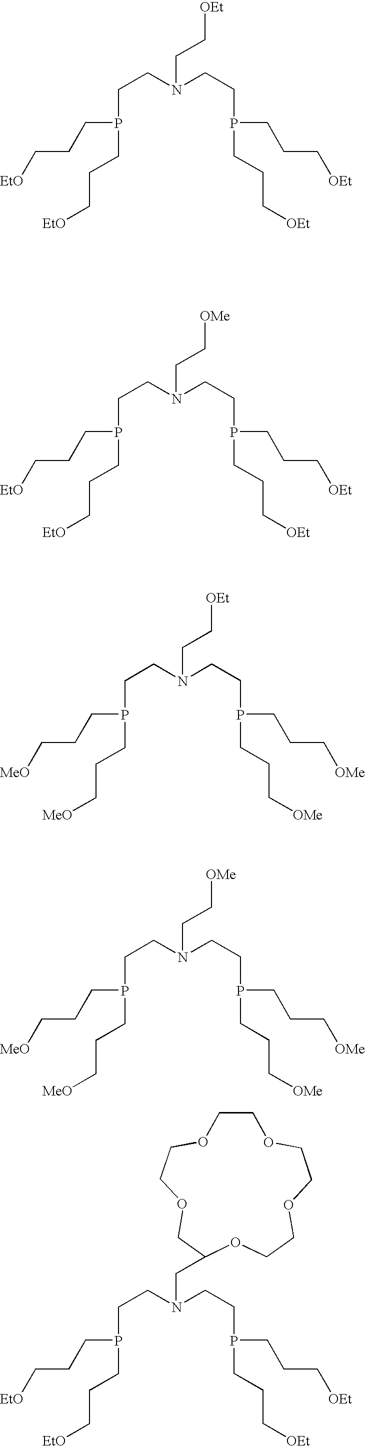

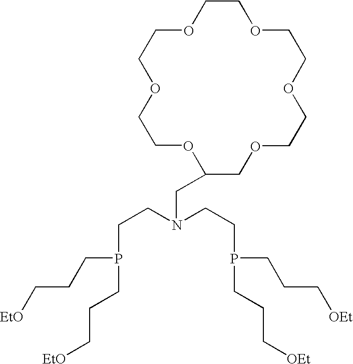

7. A crown ether-containing cationic metal complex radiopharmaceutical of claim 1, wherein L1 is selected from any one of the following crown-ether containing chelator of the formula: 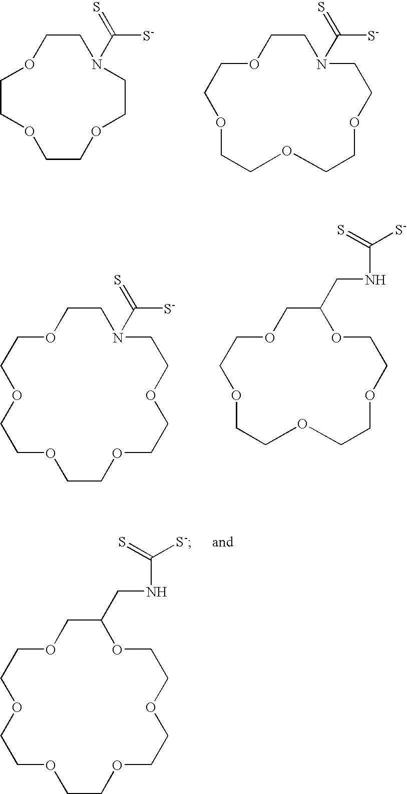
L2 is selected from any one of the following bisphosphine coligands of the formula:

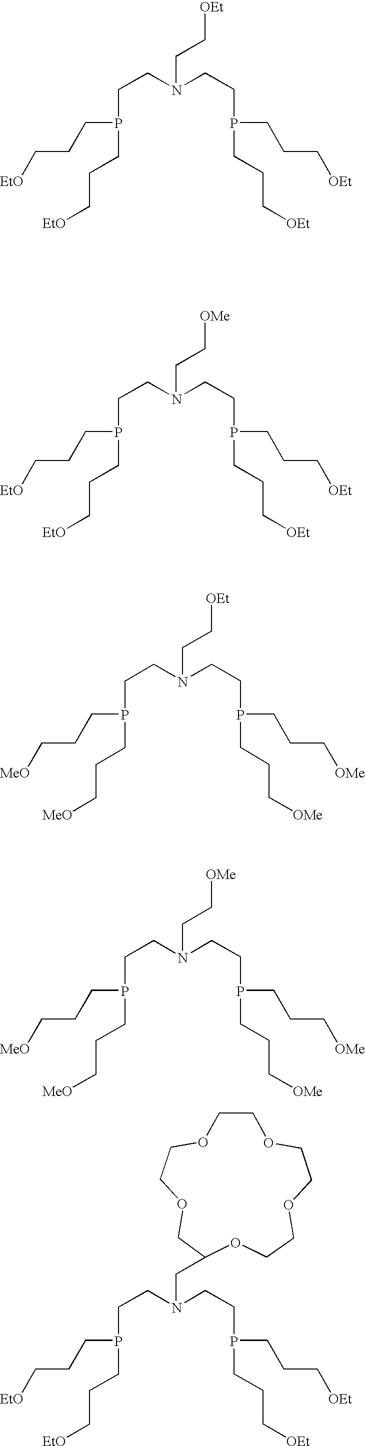

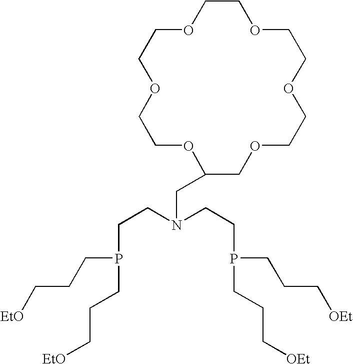

8. A novel radiopharmaceutical composition containing the radiopharmaceutical according to claim 1.

9. A method for preparation of a radiopharmaceutical product according to claim 1, comprising reacting pertechnetate with (1) a nitrido donor, (2) a reducing agent; (3) a crowned DTC chelator according to claim 1, and (4) a bisphosphine coligand according to claim 1.

10. The method of claim 9, wherein the nitrido donor is succinyl dihydride, and the reducing agent is stannous chloride.

11. A kit for preparation of a radiopharmaceutical product, comprising:
a first bottle containing a nitrido donor,
a second bottle containing a stannous chloride and a chelating agent able to stabilize the tin cation,
a third bottle containing a crowned DTC chelator according to claim 1; and
a fourth bottle containing a bisphosphine coligand according to claim 1.

12. A kit comprising, comprising:
a first bottle containing succinyl dihydride, a stannous chloride and a chelating agent able to stabilize the tin cation, and
a second bottle containing a crowned DTC chelator according to claim 1; and
a third bottle containing a bisphosphine coligand according to claim 1.

13. A Kit comprising, comprising:
a first bottle containing succinyl dihydride, stannous chloride and 1,2-diaminopropane-N,N,N′,N′-tetraacetic acid or a salt thereof, and
a second bottle containing a crowned DTC chelator according to claim 1; and
a third bottle containing a bisphosphine coligand according to claim 1.

14. A method for preparation of a radiopharmaceutical product, comprising reacting pertechnetate with (1) a diazenido donor; (2) a reducing agent; (3) a crowned DTC chelator according to claim 1, and (4) a bisphosphine coligand according to claim 1.

15. A method for preparation of a radiopharmaceutical product, comprising reacting pertechnetate with (1) a diazenido donor, (2) a reducing agent; (3) a crowned DTC chelator according to claim 1, and (4) a bisphosphine coligand according to claim 1.

16. The method of claim 15, wherein said diazenido donor is hydrazinobenzene, and said reducing agent is stannous chloride.

17. A kit for preparation of a radiopharmaceutical product comprising:
is a first bottle containing hydrazinobenzene,
a second bottle containing a stannous chloride and a chelating agent able to stabilize the tin cation,
a third bottle containing a crowned DTC chelator according to claim 1; and
a fourth bottle containing a bisphosphine coligand according to claim 1.

18. A kit for preparation of a radiopharmaceutical product comprising:
a first bottle containing hydrazinobenzene, a stannous chloride and a chelating agent able to stabilize the tin cation, and
a second bottle containing a crowned DTC chelator according to claim 1; and
a third bottle containing a bisphosphine coligand according to claim 1.

19. A kit for preparation of a radiopharmaceutical product comprising:
a first bottle containing hydrazinobenzene, stannous chloride and 1,2-diaminopropane-N,N,N′,N′-tetraacetic acid or a salt thereof, and
a second bottle containing a crowned DTC chelator according to claim 1; and
a third bottle containing a bisphosphine coligand according to claim 1.

20. A method of radioimaging a mammal comprising (i) administering to said mammal an effective amount of a radiopharmaceutical of the formula according to claim 1, and (ii) scanning said mammal using a radioimaging device.

21. A compound having the following formula: 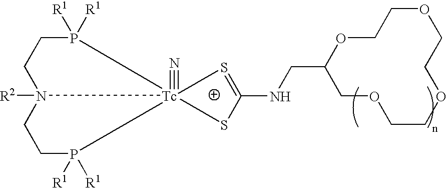
wherein R¹ is —(CH₂)₃OMe or (CH₂)₃OEt;
R² is —(CH₂)₂OMe, —(CH₂)_{OEt, CH}₂Ph, or 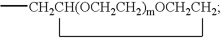
n is 2 or 3; and
m is 4 or 5.

22. A compound having the following formula: 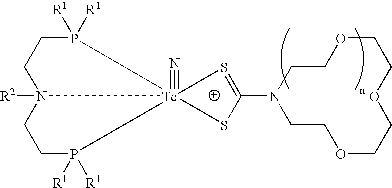
wherein R¹ is —(CH₂)₃OMe, —(CH₂)₃OEt;
R² is —(CH₂)OMe, —(CH₂)₂OEt, —CH₂Ph, or 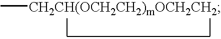
n is 1, 2, or 3; and
m is 4 or 5.
